(19) United States
(12) Patent Application Publication
(10) Pub. No.: US 2024/0360391 A1
(43) Pub. Date: Oct. 31, 2024

(54) NONWOVEN WIPES, CLEANING COMPOSITIONS, AND ASSOCIATED PACKAGING

(71) Applicant: DERMAL BIOMICS, INC., Pittsburgh, PA (US)

(72) Inventors: James NOLAN, Pittsburgh, PA (US); Michael J. FARE, Pittsburgh, PA (US); Rebecca L. BLICE, Cleveland, OH (US); Carolyn M. MCNEELEY, Cleveland, OH (US); Jess P. CARLSON, Cleveland, OH (US); Maynard PAYUMO, Cleveland, OH (US)

(21) Appl. No.: 18/526,908

(22) Filed: Dec. 1, 2023

Related U.S. Application Data
(63) Continuation of application No. 16/488,026, filed on Aug. 22, 2019, now abandoned.
(60) Provisional application No. 62/462,167, filed on Feb. 22, 2017.
(60) Provisional application No. 62/471,130, filed on Mar. 14, 2017.
(60) Provisional application No. 62/471,141, filed on Mar. 14, 2017.

(51) Int. Cl. C11D 17/04; A47K 10/32; B65D 77/04; B65D 77/38; B65D 83/08; C11D 3/386

(52) U.S. Cl. CPC C11D 17/049; A47K 10/32; B65D 77/04; B65D 77/38; B65D 83/08; C11D 3/38654; C11D 17/046; A47K 2010/3273; B65D 2203/02

(57) ABSTRACT

A single-use wipe includes a nonwoven fabric, at least one pocket defined within the fabric, the at least one pocket configured to receive at least a portion of a hand of a user of the wipe, and one or more creases defining at least one fold, wherein the wipe is configured to be folded on the one or more creases prior to being inserted into a package. The package is configured to house a burst pod containing a quantity of a fluid such as a cleanser including, for example, glucose oxidase. Prior to removal of the wipe from the package, the burst pod can be ruptured, thereby at least partially saturating the wipe with the cleanser. The then at least partially saturate wipe can be removed from the package, unfolded and used.

### CLAIM OF PRIORITY

This application claims the benefit of priority to U.S. Provisional Application No. 62/462,167, titled “Nonwoven Wipe and Packaging,” filed Feb. 22, 2017, U.S. Provisional Application No. 62/471,130, titled “Compositions and Methods for Treating Microbiota Dysregulation,” filed Mar. 14, 2017, and U.S. Provisional Application No. 62/471,141, titled “Compositions and Methods for Treating Microbiota Dysregulation,” filed Mar. 14, 2017, which are each incorporated herein by reference in their entirety.

### BACKGROUND

The present disclosure relates to disposable cloths for cleaning purposes and their associated packaging. In more specific examples, the present disclosure relates to nonwoven wipes packaged with an optional liquid contained in a burst pod such that, upon bursting the pod, a fluid is applied to the nonwoven wipes prior to removing the wipes from their packaging.

Disposable cleaning products such as pre-moistened towels have become popular for various purposes. A typical container of such disposable towels includes a stack of paper or other thin fabric towels pre-moistened with a cleaning solution, packaged in a resealable container. The container typically has a closable member such as an adhesive backed flap or a snap-shut lid configured to reseal (or partially reseal) the container, thereby increasing the time before the cleaning solution evaporates or otherwise dries on the towels.

One example of such a disposable cleaning product is commonly referred to as a baby wipe. A stack of interwoven towels are provided in a container, typically a plastic canister or box, along with a snap-shut lid. Under the snap-shut, the container typically includes a friction producing member through which the towels pass when a person removes one from the container. The friction producing member provides enough resistance that the person only removes one towel from the container at a time.

However, such existing arrangements, including the baby wipes examples as described above, typically include various drawbacks. As the container is continually opened and closed, the towels not being removed (i.e., the towels remaining in the container) are continually exposed to air and, as such, are prone to dry over time. Additionally, as the container is opened and closed repeatedly throughout its life (e.g., until all the towels contained inside are used or are otherwise unusable), the towels that remain in the container are potentially exposed to germs, either through contact with a person's skin or through airborne particles. Attempts to overcome these shortcomings still have drawbacks. For example, individually packaged premoistened wipes and/or cloths are prone to drying out over time, reducing the overall effectiveness and convenience of the single-use wipes. Similarly, a drawback to all premoistened wipes is that there is no dry option. A consumer or user of the single-use wipe may have the occasional occurrence where a dry wipe is more convenient that a moistened wipe.

Additionally, the mucosal and skin surfaces of humans are populated by a complex community of commensal organisms. These organisms provide many protective defenses to infection and disease including out-competing exogenous pathogens for nutrients. When a disruption to the commensal microbial community occurs such as with administration of antibiotics or the existence of certain co-morbidities such as diabetes, pathogens can flourish resulting in infections.

In Individuals with diabetes, the skin and mucosal surfaces can become colonized by Candida albicans and other microbes leading to painful and chronic episodes of vaginitis, balanitis, and skin infections. While Candida albicans is ubiquitous in the environment, the ingrowth of Candida into skin and mucosal surfaces can be attributed to host factors such as altered immune regulation and the production of excessive sugars in the skin and mucosa, including, for example, diabetic production of glycosurea which can act as a substrate and create a microenvironment for pathogen growth. Acute infections can be effectively treated with antimycotics as in the case of Candida, or antibiotics where the pathogen is bacterial. However, the altered physiology of the skin and mucosa creates an environment that is susceptible to future infections leading to recurrent disease, such as recurrent vulvovaginal candidiasis (RVVC) as in the case of diabetic patients.

Indeed, one of the difficulties for women with RVVC is a practice of hyper-hygenicity where the skin or mucosal surface is sterilized due to the patient repetitively cleaning the area, which, although killing the pathogens, also kills the resident beneficial microflora. The skin and mucosa thus become susceptible to the outgrowth of rapidly proliferating pathogens, while restricting the regrowth of resident commensal microflora.

As such, there is a need for a disposable cloth and/or associated packaging including compositions and methods of use that not only treat an acute infection in patients, but also diminish the likelihood of infections by preserving and promoting the growth and colonization of beneficial microflora.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a wipe is disclosed. The wipe includes a nonwoven fabric; at least one gripping feature defined within the fabric, the at least one gripping feature configured to receive at least a portion of a hand of a user of the wipe; and one or more creases defining at least one fold, wherein the wipe is configured to be folded on the one or more creases prior to being inserted into a package. In some examples, the nonwoven fabric is flushable nonwoven fabric. In some examples, the at least one gripping feature includes a pocket. In certain implementations, the pocket comprises the nonwoven fabric and, as such, is flushable. In some examples, the nonwoven fabric is dry prior to being inserted into the package and is configured to absorb a cleansing fluid. In some examples, the package is configured to hold at least one burst pod, the burst pod configured to release the cleansing fluid prior to the package being opened. In some examples, the fluid is a cleanser suitable for use on human skin. In certain implementations, the cleansing fluid comprises at least one oxido-reductase enzyme such as glucose oxidase.

In another embodiment, a package is disclosed. The package includes a burst pod at least partially attached to the package and configured to store a cleansing fluid; and a single-use wipe positioned adjacent to at least a portion of the burst pod, wherein the burst pod is configured to rupture upon application of a specific pressure, thereby releasing the cleansing fluid onto the wipe. In some implementations, the single-use wipe includes a nonwoven fabric; at least one gripping feature defined within the fabric, the at least one gripping feature configured to receive at least a portion of a hand of a user of the wipe; and one or more creases defining at least one fold, wherein the wipe is configured to be folded on the one or more creases prior to being inserted into the package. In some examples, the nonwoven fabric is flushable nonwoven fabric. In some examples, the at least one gripping feature comprises a pocket. In certain implementations, the pocket comprises the nonwoven fabric and, as such, is flushable. In some examples, the nonwoven fabric is dry prior to being inserted into the package and is configured to absorb a cleansing fluid. In some examples, the package is configured to hold at least one burst pod, the burst pod configured to release the cleansing fluid prior to the package being opened. In some examples, the cleansing fluid is a cleanser suitable for use on human skin. In certain implementations, the cleansing fluid comprises at least one oxido-reductase enzyme such as, for example, glucose oxidase. In some implementations, the package further includes at least one opening mechanism, wherein the at least one opening mechanism is a liftable flap or a tearaway strip.

### BRIEF DESCRIPTION OF THE DRAWINGS

The above and other objects of the present invention will become more readily apparent from the following detailed description taken in connection with the accompanying drawings.

FIG. 1 depicts an example disposable wipe made from a nonwoven fabric, according to one or more embodiments.

FIGS. 2A and 2B depict sample folding patterns for a disposable wipe such as that described in FIG. 1, according to one or more embodiments.

FIGS. 3A-3C depict a disposable wipe such as that described in FIG. 1, manufactured to include at least one pocket, according to one or more embodiments.

FIGS. 4A and 4B depict an alternative wipe design including at least one gripping feature, according to one or more embodiments.

FIGS. 5A and 5B depict a sample package for a disposable wipe such as those described in FIGS. 1-4B, according to one or more embodiments.

FIGS. 6A and 6B depict a sample package for a disposable wipe, the package including a liftable flap for providing access to the wipe, according to one or more embodiments.

FIGS. 7A-7D depict various examples of burst pods for use with a nonwoven wipe, according to one or more embodiments.

FIGS. 8A and 8B depict sample packages for a disposable wipe and an included burst pod, according to one or more embodiments.

FIGS. 9A and 9B depict sample packages for a disposable wipe and a burst pod integrated into the packaging material, according to one or more embodiments.

### DETAILED DESCRIPTION

The present disclosure is related to disposable cloths or wipes designed for cleaning purposes such as personal hygiene. For example, the disposable wipe can be intended for a single use to cleanse or otherwise clean areas following urination or excretion of other bodily fluids. In certain implementations, a woman (e.g., a diabetic woman) can use the wipe as described herein for vaginal wiping/cleaning following urination in an effort to prevent, for example, yeast infections.

In some examples, the wipe as described herein can be saturated with a specific composition such as glucose oxidase. As such, the present disclosure is also related to methods and compositions useful for treating infections at the mucosa or skin. Such methods and compositions comprise killing pathogenic organisms at a site of administration while preserving and enhancing the outgrowth of local protective microflora. As described herein without limitation, microbicides and fungicides can be used to prevent recurrent disease in humans and animals as well as prevent or diminish the recurrence of new infections.

Before the present teachings are described, it is to be understood that this disclosure is not limited to the particular designs, products, packages, processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must be noted that as used herein, and in the appended claims, the singular forms “a,” “an,” and “the” include plural reference unless the context clearly dictates otherwise. Thus, for example, reference to a “microbicide” is a reference to one or more microbicides and equivalents thereof known to those skilled in the art.

As used herein, all claimed numeric terms are to be read as being preceded by the term, “about,” which means plus or minus 10% of the numerical value of the number with which it is being used. Therefore, a claim to “50%” means “about 50%” and encompasses the range of 45%-55%.

“Administering” when used in conjunction with a therapeutic means to administer a therapeutic directly into or onto a target tissue, or to administer a therapeutic to a patient whereby the therapeutic positively impacts the tissue to which it is targeted. Thus, as used herein, the term “administering,” when used in conjunction with any composition described herein, can include, but is not limited to, providing microbicides and fungicide agents locally by administering such compounds into or onto the target tissue. “Administering” a composition may be accomplished by any mode including oral administration, topical administration, or by any other method known in the art. In certain embodiments, the compositions described herein may be administered in combination with another form of therapy, including, for example, systemic administration of antimycotic compounds or systemic antibiotics or any other therapy know in the art or described herein.

In certain embodiments, the compositions may be combined with a carrier. A “carrier” as used herein may include, but is not limited to, an irrigation solution, antiseptic solution, other solution, time-released composition, elution composition, bandage, dressing, colloid suspension (e.g., a cream, gel, or salve), internal or external dissolvable sutures, dissolvable beads, dissolvable sponges, and/or other materials or compositions known now or hereafter to a person of ordinary skill in the art.

The term “animal” as used herein includes, but is not limited to, humans and non-human vertebrates, such as wild, domestic, and farm animals.

The term “treating” includes the administration of the disclosed compositions thereby prevent the symptoms, alleviating the symptoms, or eliminating the disease, condition, or disorder.

By “pharmaceutically acceptable,” it is meant that the carrier, diluent, or excipient must be compatible with the other ingredients of the composition and not deleterious to the recipient thereof. By “excipient,” it is meant any inert or otherwise non-active ingredient, which can be added to the active ingredient which may improve the overall composition's properties, such as improving shelf-life, improving retention time at the application site, improving flowability, improving consumer acceptance, et alia.

Unless otherwise indicated, the term “skin” means that outer integument or covering of the body, consisting of the dermis and epidermis and resting upon subcutaneous tissue. The term “mucosa” is intended to mean any mucosal surface including the reproductive tract, the lungs, the GI tract, the oro-nasal mucosa, and transitional zones between mucosa and skin such as for example, the labia and perineum.

The term “safe and effective amount” as used herein, means an amount of an active ingredient high enough to modify the condition to be treated or to deliver the desired skin benefit, but low enough to avoid serious side effects, at a reasonable benefit to risk ratio within the scope of sound medical judgment. What is a safe and effective amount of the active ingredient will vary with the specific active, the ability of the active to penetrate through the skin, the age, health condition, and skin condition of the user, and other like factors.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to kill or prevent the growth of pathogen(s) while preserving one or more species of local microflora. For example, a therapeutic effect may be demonstrated by increased killing of a pathogen, growth reduction of a pathogen, decreased proliferation of pathogens such as bacterio- or myco-stasis, increased proliferation of commensal microflora, increased colonization rates of commensal microflora, reduction of symptoms, and sequellae as well as any other therapeutic effect known in the art.

The term “commensal” is intended to include any resident beneficial prokaryotic, archaebiotic, or eukaryotic microorganism. As such, in a human it is recognized that many different commensal species exist and such microflora live in particular niches suitable for colonization. For example, the normal commensal population of microbes participates in the metabolism of food products, provides essential growth factors, protects against infections with highly virulent microorganisms, and stimulates the immune response. The microbial flora in and on the human body is in a continual state of flux determined by a variety of factors, such as age, diet, hormonal state, health, and personal hygiene. Throughout the life of an individual, this microbial population continues to change.

As noted above, the present disclosure is related to disposable cloths or wipes designed for cleaning purposes such as personal hygiene. In certain examples, the disposable wipe can be manufactured from a specific material such as a nonwoven fabric. In some implementations, the wipe can be designed and manufactured such that one or more pockets are included therein, located such that a user of the disposable wipe can place one or more fingers into the pockets to better control the wipe during the cleaning process. In some examples, the packaging for the disposable wipe is designed such that the one or more pockets are easily accessible by the user without contaminating or otherwise touching other portions of the wipe, thereby reducing or eliminating the potential for spread of germs from the user's hands to other areas of their body being cleaned with the wipe.

In certain implementations, a burst pod filled with a cleaning agent or other similar fluid can be included. In some examples, the burst pod can be included in a package with a dry disposable wipe. The burst pod can be configured to rupture upon application of a specific pressure or force, thereby releasing the fluid contained within the burst pod into the packaging for absorption by the previously dry wipe. Such an arrangement overcomes various drawbacks of the prior art, specifically that the wipe is moistened prior to insertion into the package. Such a design could result in pocket or feature degradation due to constant exposure to the moisture, or degradation to the cloth itself that could be caused by extended exposure to the leaning agent. Such a drawback is not present with the invention as described herein as the wipe, along with its various features such as the pockets, are not exposed to any fluids until the burst pod is ruptured, thereby maintaining structural integrity of the pocket and/or cloth wipe.

In some examples, prior to opening the package, a person can rupture the burst pod (e.g., through application of an adequate force), thereby releasing a cleaning fluid from inside the burst pod. The fluid can penetrate the dry disposable wipe, thereby dampening the wipe with the released fluid. Depending upon the volume of liquid contained in the burst pod, the placement of the burst pod, and the size and shape of the wipe, saturation of the wipe itself can vary between designs. For example, the burst pod can contain enough fluid to partially wet the wipe, providing a dry area and a wet area. For convenience of use, the burst pod can be positioned such that a pocket and the area immediately surrounding the pocket remain dry or relatively dry such that a person can easily access the pocket and place, for example, one or more fingers into the pocket. As such, after rupturing the burst pod, the person opens the package, removes the now-dampened wipe and proceeds to use the wipe for one or more cleaning purposes.

The examples and implementations as described above are further illustrated in greater detail in the following discussion of the accompanying figures. However, it should be noted that while the following discussion is typically directed to disposable wipes and accompanying packaging intended for cleaning purposes such as personal hygiene, the ideas and techniques described herein are applicable to any scenario where, for example, a single-use disposable wipe would be desirable.

As described herein, the disposable wipes can be manufactured or otherwise made from a material designed to be easily disposed of via, for example, flushing. The disposable wipe can include a single or multi-layered structure that is made with materials having a general thickness of, in some examples, between 0.001 and 0.125 inches. In certain implementations, the disposable wipe can include multiple sides. For example, one side can be configured as an application side configured to be saturated with a liquid such as a cleaning solution. In certain implementations, a second side can be configured to be a handling side including, in some examples, one or more pockets or coverings configured to receive at least a portion of a user's hand (e.g., one or more fingers), covering that portion of the user's hand during use of the disposable wipe.

As noted above, the disposable wipe as described herein can be made from a flexible material such as nonwoven fabrics. Nonwoven fabric is typically a fabric-like material made from long fibers that are bonded together by chemical, mechanical, heat or solvent treatment. The fibers are typically made from separate plant fibers, molten plastic, plastic films, or a combination of materials. Resulting nonwoven fabrics can be flat or tufted porous sheets that are made directly from bonding the individual fibers. Based upon the manufacturing process and the type of bonding used, nonwoven fabrics can degrade quickly when compared to woven fabrics. For example, by using a bonding process that is broken down by application of a liquid such as water, a nonwoven fabric can be disposed of by flushing the fabric down a standard toilet. Similarly, an adhesive material that is broken down and quickly dispersed by application of a liquid such as water can be used to in the bonding process to form the nonwoven fabric.

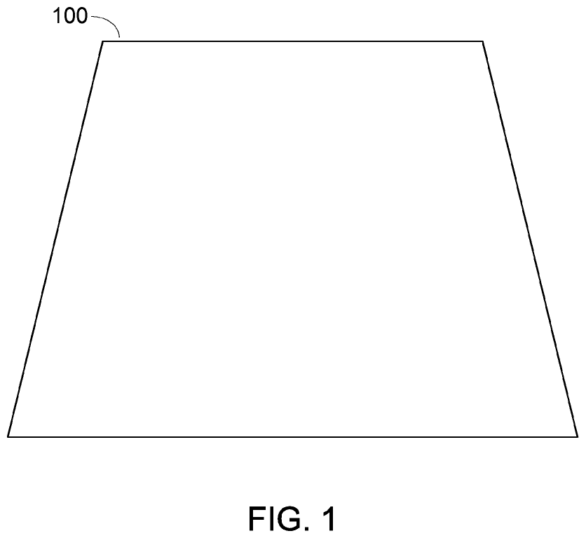

The following description and accompanying figures describe various features of the disposable wipe as taught herein. For example, FIG. 1 illustrates a view of a wipe 100. As noted above, wipe 100 can be manufactured from a nonwoven fabric. In certain implementations, the nonwoven fabric can include a pattern or texture. For example, the wipe 100 can be manufactured to have a crosshatched texture, a striped or rippled texture, a wavy texture, or a combination of these or other similar texture styles and patterns.

Depending upon the intended use of the wipe, the shape and size of the wipe 100 can vary. For example, the wipe 100 can be shaped as a square or rectangle (similar to that as shown in FIG. 1); a circle, oval or other similar rounded shape; a triangle, pentagon, or other similar regular geometric shape; a custom shape such as a hand or other body part; or any other shape dependent upon the design and intended use of the wipe. Similarly, the size of the wipe 100 can vary depending upon the intended use. In certain implementations, the size of the wipe 100 can be about 7 inches by 7 inches. In some examples, the size of the wipe can be about 7.875 inches by 6.375 inches. In other implementations, the size of the wipe 100 can be about 10 inches by 10 inches. In some examples, the wipe 100 can be manufactured and sold in various sizes. For example, a small size (e.g., about 6 inches square), a medium size (e.g., about 9 inches square), and a large size (e.g., about 12 inches square) can all be manufactured and sold in, for example, a single variety pack (e.g., including a certain number of each size) or in single size packages.

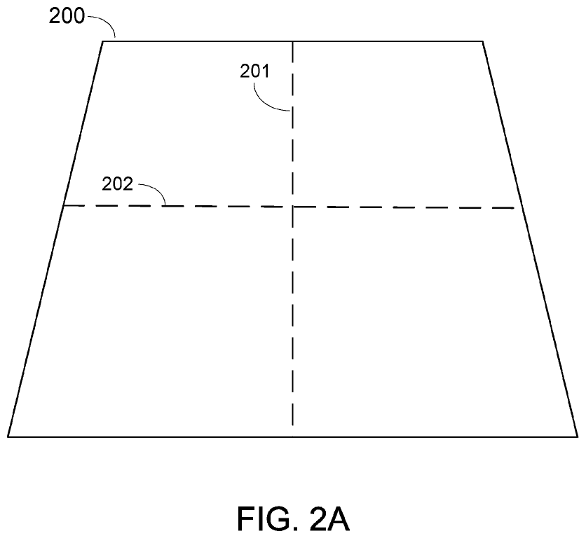

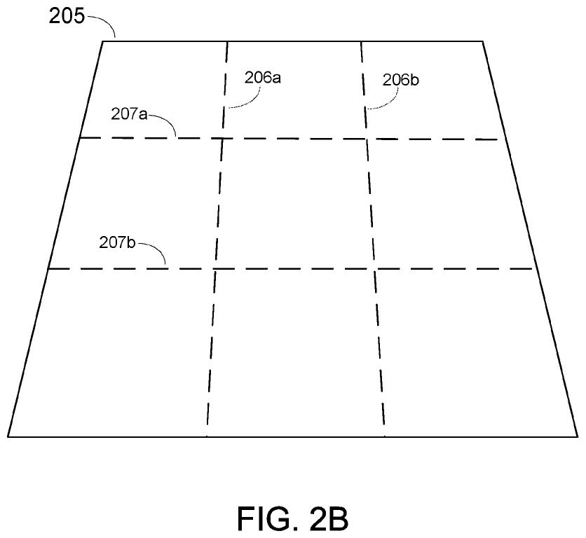

Depending upon the shape and size of the wipe 100, the wipe 100 can be folded prior to and for insertion into an appropriate package. For example, as shown in FIG. 2A, a wipe 200 can be folded twice (e.g., along fold lines or creases 201 and 202), resulting in a four layered wipe when the wipe is inserted into a package. In a second example, as shown in FIG. 2B, a wipe 205 can be folded four times, twice vertically (e.g., along fold lines or creases 206a and 206b), and twice horizontally (e.g., along fold lines or creases 207a and 207b). This arrangement as shown in FIG. 2B would result in a 9 layered folded wipe when the wipe 205 is inserted into a package.

It should be noted that the examples as shown in FIGS. 2A and 2B are provided for illustrative purposes only. The actual folding technique as used when preparing a wipe for packaging can be determined according to various factors such as the overall size of the wipe as compared to the size of the package, the shape of the wipe as compared to the shape of the package, the desired thickness of the wipe and/or package when the wipe is folded (e.g., how many layers thick can/should the package be when the folded wipe is inserted), and other similar factors. In certain implementations, the wipe can be folded into various geometric shapes such as a triangle, a circle, a square, a rectangle, and other similar multi-sided shapes and polygon, as well as decorative shapes such as a flower.

Additionally, in some examples, the wipe can be folded such that it maintains its folded shape until a force is exerted on the wipe, such as a person purposely unfolding the wipe. In certain implementations, in order to maintain this folded shape or configuration, an adhesive such as the adhesive used to bond the nonwoven fibers can be used in a lesser quantity or adhesive strength such that the wipe can be unfolded. In some examples, an alternative mechanism can be used to maintain the folded shape or configuration such as stapleless staples, stitching using dissolvable thread or a similar nonwoven material as the wipe is formed from, and other non-adhesive mechanisms. For example, the order of folding the wipe can be set such that the stacking and friction of adjacent layers holds the wipe in the folded configuration. Regardless of the techniques used to maintain the folded shape and configuration, the flushability and ease of disposal of the wipe remains unchanged.

In certain implementations, the disposable wipe as described herein can include additional features to increase its efficiency when used, for example, as a personal hygiene wipe. In such an application, a user might want a protective feature to at least partially cover a portion of their hand during use. The protective feature or cover can also improve a person's feel when using the wipe, allowing the user to more accurately position the wipe and vary any applied pressure exerted on the wipe as appropriate during use.

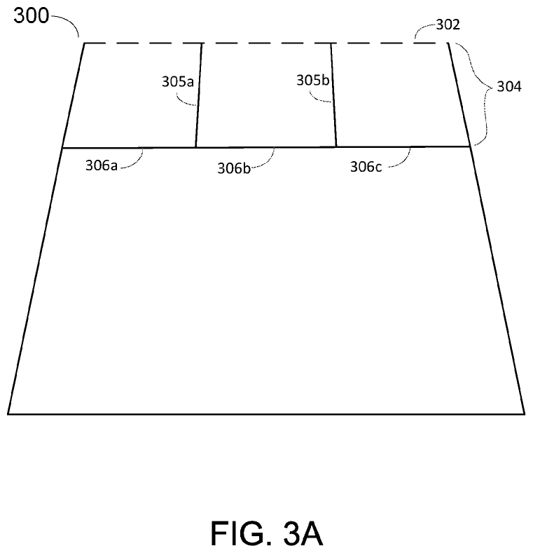

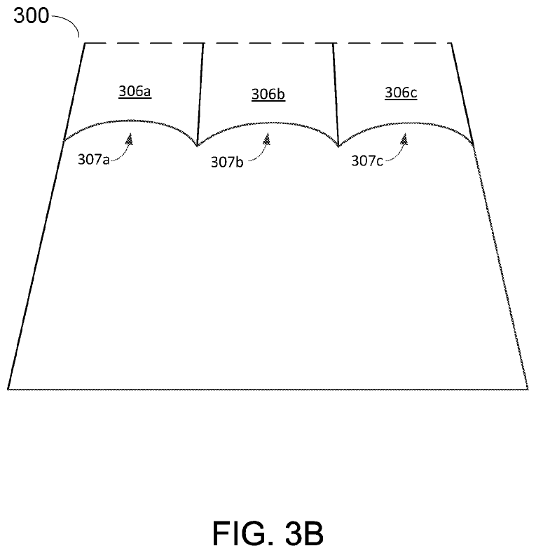

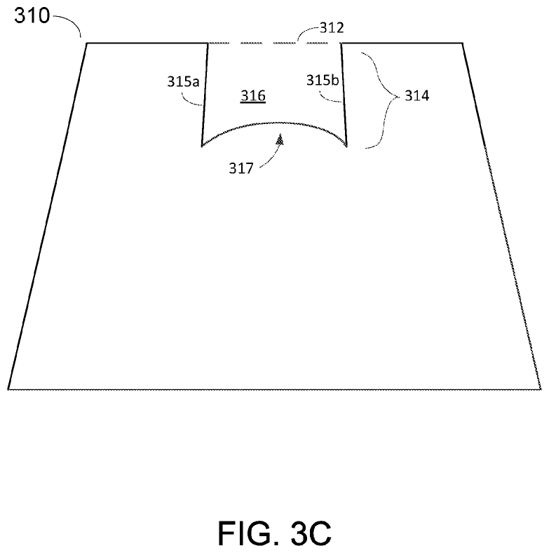

For example, FIG. 3A illustrates a wipe 300 that includes a series of pockets for covering at least a portion of the user's hand when using the wipe. For example, as shown in FIG. 3A, a set of three pockets 306a, 306b and 306c are defined and included on wipe 300. In certain implementations, the pockets 306a, 306b, and 306c can be manufactured from the same material as the wipe 300, thus maintaining the wipe's disposable qualities such as flushability. For example, if the wipe 300 is manufactured from a flushable nonwoven fabric, the addition of the pockets 306a, 306b, and 306c as described herein would not alter the flushability of the wipe.

As shown in FIG. 3A, a portion 304 of the wipe 300 can be folded back over onto the top of the wipe at crease 302. Depending upon the size and intended use of the wipe 300, the folded portion 304 can vary in size. For example, the folded portion 304 can be a certain percentage of the total size of the wipe 300 (e.g., 33% or 50%). In some examples, the folded portion 304 can be sized such that it defines a specific sized pocket that is sized and configured to receive a portion of a person's hand such as one or more fingers. For example, the folded portion 304 can be sized such that the resulting pockets 306a, 306b, and 306c are sized about 3 inches deep to receive at least a portion of a user's finger.

The folded portion 304 of wipe 300 can also include multiple delineation lines that further provide a definition of the sides of each of the pockets 306a, 306b, and 306c. For example, as shown in FIG. 3A, two lines 305a and 305b can be included, thereby defining the three pockets 306a, 306b, and 306c in the folded portion 304. The lines 305a and 305b can be made using a similar bonding technique as that used to make the nonwoven fabric for the wipe 300. For example, a chemical, heat, or solvent treatment can be applied at lines 305a and 305b, thereby adhering the nonwoven fabric of folded portion 304 to the nonwoven fabric underneath. For example, two metal dies including heated metal strips sized and positioned similar to 305a and 305b as shown in FIG. 3A can be pressed against wipe 300, thereby bonding the folded portion 304 to the portion underneath, thereby defining the pockets 306a, 306b, and 306c.

As shown in FIG. 3B, the pockets 306a, 306b, and 306c can be opened, thereby defining empty spaces 307a, 307b and 307c, proving spaces for at least a portion of the user's hand. For example, when considering wipe 300 as shown in FIGS. 3A and 3B, a user can use their right hand to use wipe 300. The user can insert their right pointer finger into pocket 306a (thereby occupying at least a portion of space 307a), the user can insert their right middle finger into pocket 306b (thereby occupying at least a portion of space 307b), and the user can insert their right ring finger into pocket 306c (thereby occupying at last a portion of space 307c). The user can then move their inserted fingers separately or in concert during use of the wipe 300, improving the functionality of the wipe as compared to standard wipes by improving the user's control and ability to accurately apply pressure and the resulting wiping force.

It should be noted that three pockets as shown in FIGS. 3A and 3B are shown by way of example only. It certain implementations, alternative numbers of pockets can be used. For example, as shown in FIG. 3C, wipe 310 can include a single pocket 316. The wipe includes a narrower folded portion 314 (when compared to wipe 300 and folded portion 304). The folded portion 314 can be folded at crease 312, thereby overlapping at least a portion of the wipe 310. The folded portion 314 can be adhered to the portion of wipe 310 underneath the pocket at, for example, lines 315a and 315b. Similar to above, the lines 315a and 315b can be made using a similar bonding technique as that used to make the nonwoven fabric for the wipe 310. Also like above, pocket 316 can be opened, thereby defining empty space 317. A user, when using wipe 310, can insert one or more fingers into the pocket 316, thereby occupying at least a portion of empty space 317. For example, the user can insert their right middle finger into pocket 316, providing the user with improved control over the wipe 310 when compared to traditional wipes without pockets.

The pocket examples as shown in FIGS. 3A-3C are shown by way of illustrative purposes only. Alternative pocket arrangements, numbers and shapes can be implemented based upon various factors including, but not limited to, the size and shape of the wipe, the type of packing used, the intended use of the wipe, and other similar factors. For example, if the wipe is circular in shape, a semicircular pocket can be included that covers, for example, half of the circular wipe. In another example, if the wipe is shaped similar to a human hand, the wipe can include pockets at each of the hands fingertips such that a user can wear the wipe similar to a glove.

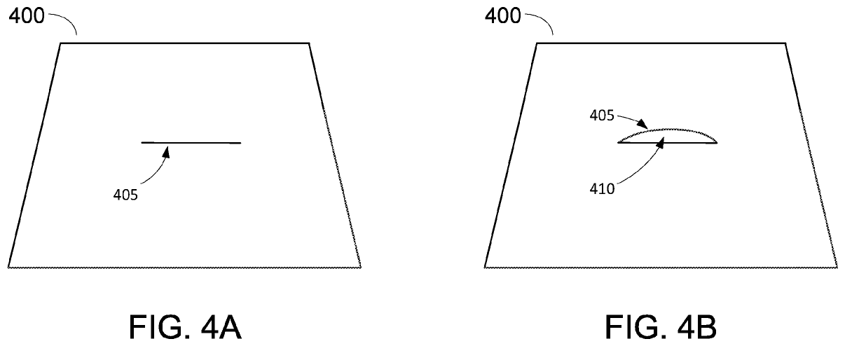

FIGS. 4A and 4B illustrate an additional nonwoven wipe design that includes an alternative gripping feature. Rather than a pocket as shown, for example, in FIGS. 3A-3C, a wipe 400 can include a cut 405, slit, or other perforation in the material. The cut 405 can be configured to form a hole 410, as shown in FIG. 4B, when pulled away from or otherwise disengaged from the surrounding wipe material. Such a hole 410 can provide a gripping or access point for a portion of the user's hand to access when using the wipe. For example, a person using wipe 400 can slide a portion of their hand such as one or more fingers into the hole 410 to provide better grip on the wipe as well as to provide a pressure applying point (e.g., the portion of the hand inserted into the hole) for using the wipe.

It should be noted that a single, centered cut 405 is shown in FIGS. 4A and 4B by way of example only. In certain implementations, a wipe such as wipe 400 can include two or more cuts, slits, or other perforations configured to provide a gripping or access point for at least a portion of a person's hand.

As noted above, the wipes as described in reference to FIGS. 1-4B are configured to be inserted into a package such that, in certain implementations, each package includes one single-use wipe. It should be noted that, while the following description refers to packaging for one single-use wipe, the packaging styles and techniques as described herein can be applied to packages for multiple single-use wipes.

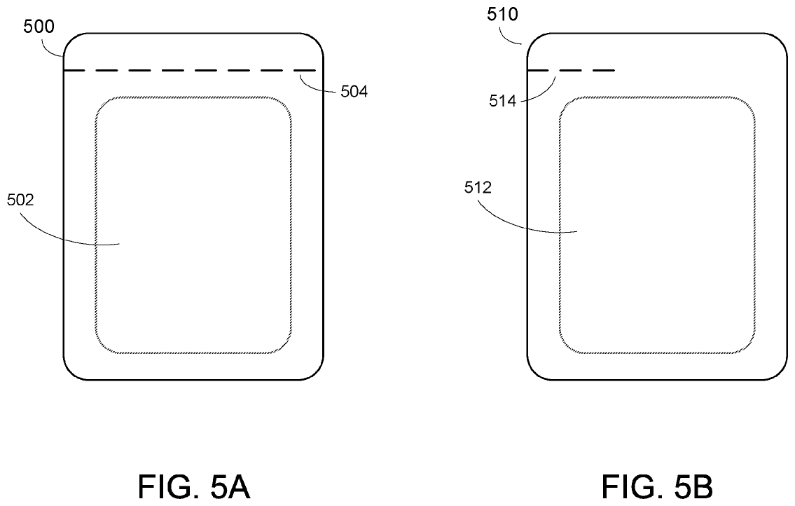

FIGS. 5A and 5B illustrate a sample package configured to hold at least one of the disposable wipes as described above. As shown in FIG. 5A, a package 500 can be sized and shaped to hold a wipe 502. Depending upon the size, shape, and number of folds, the size of wipe 502 when inserted into package 500 can vary. For example, as shown in FIG. 2A, a twice folded wipe resulting in a 4 layered wipe can be inserted into the package, the resulting package having a thickness of two times the packaging material plus four times the thickness of the disposable wipe. In an alternate example, as shown in FIG. 2B, a four-time folded wipe resulting in a 9 layered wipe can be inserted into the package. The resulting package would have a thickness of two times the packaging material plus nine times the thickness of the disposable wipe.

Referring again to FIG. 5A, the package 500 can also include a feature for facilitating easy opening of the package. For example, the package 500 can include a perforated line 504 that defines a position where the package is weakened during the manufacturing process for easier opening of the package. It should be noted that the perforate line 504 can be positioned such that it is away from the wipe 502 when the wipe is inserted into the package 500. Such an arrangement reduces the chance of a person ripping or otherwise damaging the wipe 502 when opening the package 500.

It should be noted that a full width perforated line 504 such as that shown in FIG. 5A is shown by way of example only. In certain implementations, such as that shown in FIG. 5B, a package such as package 510 can include a partial perforated line 514 that only spans a portion of the width of the package. In this example, the wipe 512 can also be similarly positioned within the package 510 away from the partial perforated line 512, thereby reducing the chance that a person ripping the package open would damage the wipe prior to use.

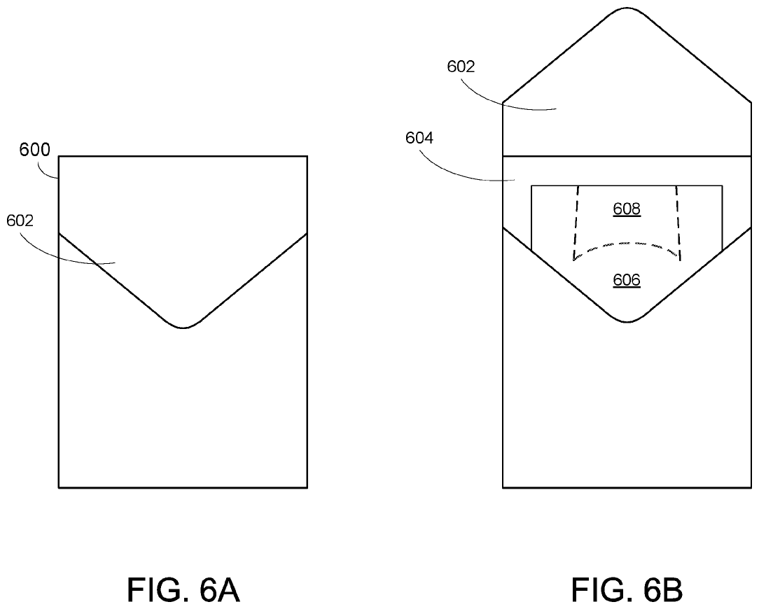

FIGS. 6A and 6B illustrate an alternative package design for a single-use wipe. As shown in FIG. 6A, a package 600 can include a liftable flap 602 configured to seal a single-use wipe within package 600. To open, a person can lift on an end of the liftable flap 602, thereby opening a portion of the package (e.g., open portion 604 as shown in FIG. 6B). The open portion 604 can include access to at least a portion of a single use wipe 606. As shown in FIG. 6B, the wipe 606 can be positioned such that at least a portion of a pocket 608 is exposed when liftable flap 602 is lifted, thereby providing a person opening the package with convenient access to the wipe. In certain implementations, the person can insert one or more fingers into pocket 608, slide the wipe 606 out of the package 600, unfold the wipe and use the wipe as they intend. It should be noted that pocket 608 is provided by way of example only, and depending upon the design and manufacture of wipe 606, the wipe may not include a pocket at all, or may include a gripping or access point such as those described above in regard to FIGS. 4A and 4B.

It should be noted that various details such as size, shape, and wipe placement as shown in FIGS. 5A-6B are shown by way of example only. The size and shape of the packages as shown in FIGS. 5A-6B can be varied based upon numerous factors. For example, the packages can be sized or shaped to appropriately fit the wipe (e.g., having a similar size and shape as the wipe) without resulting in too much waste or unutilized packaging material. In certain examples, the packages can be shaped to resemble a familiar object such as a flower, or include graphics, lettering, or other similar physical indicia representative of the product, brand, and/or manufacturer.

It should also be noted that the example opening mechanisms (e.g., a perforated tear line and a liftable flap) are shown by way of example only. Depending upon the design of the packages, additional opening mechanisms such as a resealable opening mechanism, a pull apart seam, a blister pack arrangement, and other similar opening mechanisms can be used.

Depending upon the intended use of the wipe, and the condition of the wipe when inserted into the package (e.g., dry, partially moistened, fully saturated), the package as described above can be made from various materials. For example, the package can be made from a coated paper that has a low water permeability rating on at least one side that is to be positioned adjacent to the wipe. The package can also be manufactured from a material such as foil, foil-lined paper, a plastic resin such as an ionomer resin, and other plastics such as polypropylene. In certain implementations, the package can be manufactured from a clear or semi-transparent material. In some examples, if the wipe and/or cleanser contained within the package are sensitive to ultraviolet radiation, the package material can be opaque.

As noted above, in certain implementations, a burst pod can contain a quantity of fluid such as a liquid cleanser. In some examples, a single-use wipe can be packaged along with a burst pod. Prior to use, the burst pod can be ruptured or otherwise compromised and, upon rupturing the burst pod, the fluid contained therein is released, the released fluid at least partially saturating the wipe. The package can then be opened, and the at least partially saturate wipe can be removed and used.

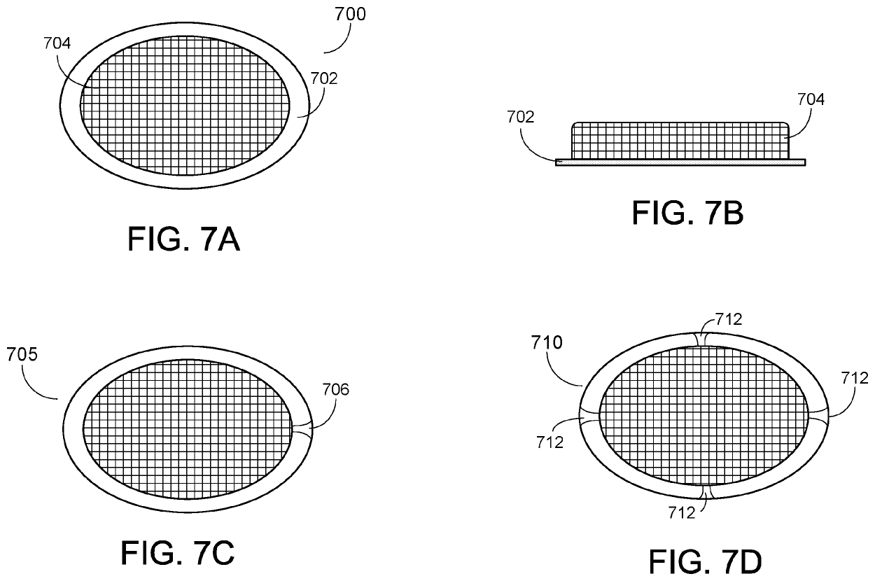

FIGS. 7A-7D illustrate example views of various sample burst pods. FIG. 7A illustrates a sample burst pod 700 for including, for example, in a package along with a single-use wipe such as those described herein. The burst pod 700 can include, for example, a base 702 that is configured to be adhered or otherwise fastened to a portion of the package. In certain implementations, the burst pod can also be integrated directly into the package. The burst pod can also include a reservoir 704 filled with, for example, a fluid such as a liquid cleanser. The reservoir 704 can be manufactured and configured such that, upon application of a sufficient pressure, the reservoir ruptures or otherwise fails, thereby releasing the fluid contained therein. FIG. 7B illustrates a sample side-view of the burst pod 700, including the base 702 and the reservoir 704.

The burst pod 700 can be manufactured from a plastic such as an ionomer resin molded in sheets. The base 702 can be manufactured from a thicker material that the reservoir 704 such that the base provides some rigidity for mounting the burst pod 700 to the package as well as forms a barrier to direct the fluid away from the base when the reservoir is ruptured. In certain implementations, the reservoir 704 can be molded, filled with the fluid, and adhered to the base 702 using, for example, application of heat and pressure to form a bond between the materials used to make both the base and the reservoir. In certain implementations, a reservoir can be molded using a form, fill and seal process. The reservoir can be formed from, for example, a resin film, thereby defining a pocket to receive the fluid. The fluid can be filled into the reservoir pocket, and the pocket can be sealed (e.g., through application of heat and pressure), thereby forming a complete reservoir of fluid for the burst pod.

The material used to make the reservoir 704, as well as the bond between the reservoir and the base 702, should be strengthened such that accidently rupture of the reservoir (e.g., from carrying a package including the burst pod in a pocket or purse) is unlikely. For example, the reservoir 704 can be designed such that an application of 10 psi of pressure is required to rupture the burst pod. In certain implementations, the reservoir 704 can be configured to hold a specific quantity of fluid such as a gentle cleanser for use on human skin without adversely impacting the human skin. For example, the reservoir 704 can be configured to hold approximately 5-10 ml of fluid. In some implementations, the reservoir 704 can be configured to hold approximately 7.5 ml of fluid. In some examples, the reservoir 704 can be configured to hold approximately 25 ml of fluid. In some implementations, the reservoir 704 can be configured to hold approximately 15-50 ml of fluid.

It should be noted that including a base 702 as is shown in FIGS. 7A and 7B is by way of example only. In some implementations, the burst pod can simply include a fluid reservoir such as reservoir 704 directly attached to a side of the package without including an additional component such as the base. In such an implementation, the base as shown in FIGS. 7A and 7B can be replaced by a portion of the package itself, and the reservoir would be adhered directly to the package. However, depending upon the materials used and the strength of the reservoir, including an additional component such as a base can increase the overall strength of the burst pod. In other examples, as noted above, the reservoir of the burst pod can be integrated directly into the package itself. In such an example, the reservoir can be formed when the package is manufactured, the reservoir filled and sealed prior to insertion of the wipe, and the wipe inserted into the package and the package sealed. In such an example, the exterior package can be reinforced to better protect the integrated reservoir. For example, an area adjacent to and/or opposite of the burst pod can be reinforced to reduce the chance of accidental rupturing of the burst pod.

It should also be noted that the burst pod is shown as having a round shape by way of example only. Alternate shapes such as rectangles, triangles, and other similar geometric shapes can be used. In certain implementations, the shape of the burst pod can be determined based upon the shape and size of the package the burst pod is being integrated into.

In some implementations, the burst pod can be adhered to the side or a corner of the package using one or more adhesion techniques. For example, the burst pod can be adhered to the package using an adhesive such as glue or epoxy. In another example, the burst pod can be adhered to the package using a chemical or heat/pressure bonding technique. Depending upon the size and shape of the burst pod, various portions of the burst pod can be attached to the package. For example, if the burst pod is rectangular shaped, an edge or seam of the burst pod can be adhered to a portion of the package. In certain implementations, the package material can be reinforced adjacent to where the burst pod is adhered such that the burst pod is less likely to accidentally rupture. Regardless of the adhesion technique, the burst pod can be configured to maintain its attachment to the package throughout the complete use cycle of the package, burst pod and single-use wipe as described herein.

Depending upon which portion of the reservoir 704 fails, dispersal of the fluid contained there can be potentially unpredictable. In a small package having a wipe with a smaller surface area, this may not be a concern. Similarly, when using a large reservoir containing a larger quantity of fluid, this may not be a concern. However, in order to maximize efficiency of both the wipe and the reservoir, the burst pod can be designed to include one or more designed exit ports configured to direct the flow of the fluid when released from the reservoir.

FIG. 7C illustrates a burst pod 705 that includes at least one exit port 706 designed to direct the flow of the fluid from the reservoir to the wipe. Similarly, FIG. 7D illustrates a burst pod 710 with multiple exit ports 712 arranged to provide a more distributed dispersal of the fluid upon rupturing of the burst pod. Such arrangements provide for a more controlled release of the fluid, providing the manufacturer of the product to design the burst pad and wipe combination such that the wipe is more thoroughly saturated upon rupturing of the reservoir on the burst pod.

In certain implementations, the burst pod can be further designed to fail in a predictable manner. For example, an edge or seam on the burst pad can be designed such that it is weaker than the rest of the burst pod. In such an arrangement, the burst pod is likely to fail at the weakened point, and dispersal of the fluid contained within the burst pod can be directed by positioning the weakened point at a particular location within the package.

As noted above, and shown in FIGS. 7A and 7B, the burst pod can include a base configured to be attached to the package. By providing for an attachment to the package, the burst pod can be contained within the waste packaging once the wipe has been removed from the package. As noted above, in certain implementations, the wipe is manufactured from a nonwoven fabric that is configured to be flushable. By securely attaching the burst pod to the package, the only component that is removed from the package is the wipe and, as such, all material removed from the package can be easily disposed of, e.g., by flushing. This also provides for an arrangement where the package and the burst pod can be manufactured from a more substantial material that is not flushable. Rather, the package and burst pod can be manufactured from a material that is to be recycled or disposed of in a solid waste trash receptacle such as a bathroom garbage can.

In certain implementations, a burst pod such as those shown in FIGS. 7A-7D can be sized to hold a specific amount of fluid and to fit on or within a certain package. For example, the burst pod can be about 65 mm in length. In certain implementations, the burst pod can be about 50 mm to about 80 mm in length. In an example, the burst pod can be about 50 mm in width. In certain implementations, the burst pod can be about 40 mm to about 60 mm in width. In an example, the burst pod can be about 11 mm tall. In certain implementations, the height of the burst pod can be about 9 mm to about 13 mm.

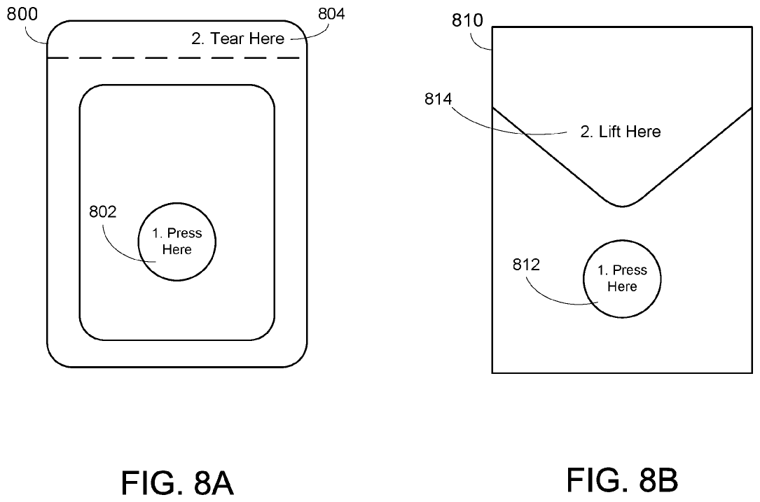

FIGS. 8A and 8B illustrate sample packages including both a nonwoven wipe as well as a burst pod. As shown in FIGS. 8A and 8B, the location of the burst pod can be highlighted by a symbol or text indicating that the person is to push at that spot (or perform some other action to rupture the burst pod). For example, as shown in FIG. 8A, a single-use wipe package 800 can include both a folded nonwoven wipe and a burst pod. The package 800 can include graphics, wording, or other indicia providing instructions for the person to properly open the package. For example, as shown in FIG. 8A, the package 800 can include a first instruction 802 that says “1. Press Here,” indicating where the person is to apply pressure to rupture the burst pod. The package 800 can further include a second instruction 804 that says “2. Tear Here,” indicating an opening mechanism for removing the wipe from the package, i.e., a perforated tear line similar to that as described above in reference to FIG. 5A.

Alternative types of packaging can have similar instructions. For example, FIG. 8B illustrates a single-use wipe package 810 that includes a liftable flap such as those described above in reference to FIGS. 6A and 6B. Like above, the package 810 can include a first instruction 812 that says “1. Press Here,” indicating where the person is to apply pressure to rupture the burst pod. The package 810 can further include a second instruction 814 that says “2. Lift Here,” indicating an opening mechanism for removing the wipe from the package, i.e., the liftable flap.

In certain implementations, a package such as package 800 as shown in FIG. 8A can be sized to receive both an appropriately sized wipe as well as to hold a particular amount of fluid in an integrated burst pod. For example, package 800 can be approximately 110 mm in length. In certain implementations, package 800 can be about 90 mm to about 130 mm in length. In an example, the width of package 800 can be about 90 mm. In certain implementations, the width of package 800 can be about 75 mm to about 105 mm.

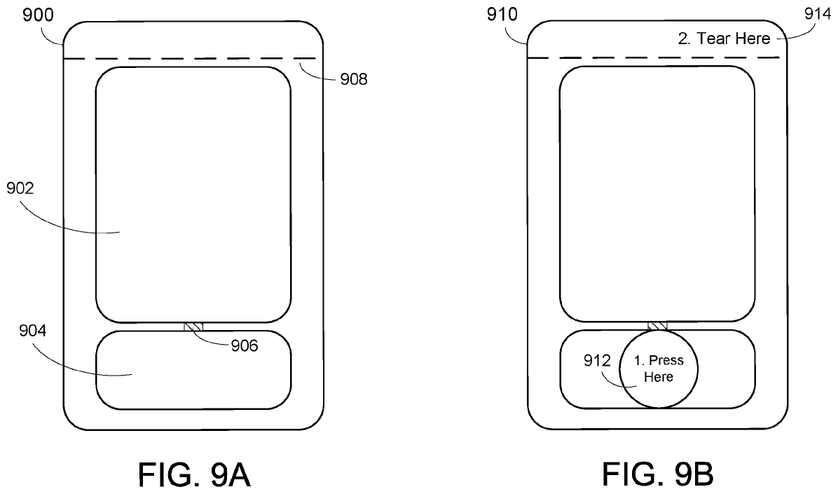

The packaging as shown in FIGS. 8A and 8B includes a burst pod that is at positioned over or otherwise directly adjacent to at least a portion of a disposable wipe. However, this arrangement is shown by way of example only, and other arrangements can be included in a single-use wipe and packaging. For example, as shown in FIG. 9A, a package 900 can be sized and shaped to hold a wipe 902. As noted above, depending upon the size, shape, and number of folds, the size of wipe 902 when inserted into package 900 can vary. The package 900 can also include an integrated reservoir 904 configured to hold an amount of a fluid such as a cleaners as described herein. The reservoir 904 may be sealed within the package 900 such that the fluid is maintained in a separate location and isolated from the wipe 902 until a person is ready to the use the wipe. The person can then apply a certain pressure to the reservoir 904, thereby causing failure and rupture of a frangible portion 906 of the package. Once the frangible portion 906 has ruptured, the fluid can flow from the reservoir 904 to wipe 902, thereby saturating the wipe. The package 900 can also include a feature for facilitating easy opening of the package. For example, the package 900 can include a perforated line 908 that defines a position where the package is weakened during the manufacturing process for easier opening of the package. It should be noted that the perforate line 908 can be positioned such that it is away from the wipe 902 when the wipe is inserted into the package 900. Such an arrangement reduces the chance of a person ripping or otherwise damaging the wipe 902 when opening the package 900. The perforate line 908 can also be positioned such that it is away from the reservoir 904, thereby reducing a chance of the fluid being accidentally or inadvertently released from the reservoir.

It should be noted that a full width perforated line 908 such as that shown in FIG. 9A is shown by way of example only. In certain implementations, the package 900 can include a partial perforated line that only spans a portion of the width of the package.

In certain implementations, the package 900 as shown in FIG. 9A can be sized to receive both an appropriately sized wipe 902 as well as to hold a particular amount of fluid in reservoir 904. For example, package 900 can be approximately 125 mm in length. In certain implementations, package 900 can be about 100 mm to about 150 mm in length. In an example, the width of package 900 can be about 75 mm. In certain implementations, the width of package 900 can be about 50 mm to about 100 mm. In an example, reservoir 904 can be configured to hold about 7.5 ml of fluid. In certain implementations, reservoir 904 can be configured to hold about 5 ml to about 10 ml of fluid.

As shown in FIG. 9B, a single-use wipe package 910 can include both a folded nonwoven wipe and fluid reservoir such as those described in reference to FIG. 9A. The package 910 can include graphics, wording, or other indicia providing instructions for the person to properly open the package. For example, as shown in FIG. 9B, the package 910 can include a first instruction 912 that says “1. Press Here,” indicating where the person is to apply pressure to rupture the frangible portion between the fluid reservoir and the wipe. The package 800 can further include a second instruction 914 that says “2. Tear Here,” indicating an opening mechanism for removing the wipe from the package, i.e., a perforated tear line similar to that as described above in reference to FIG. 9A. Depending upon the amount of fluid included in the reservoir, the type of wipe used, and the overall design of the package, additional instructions such as shake package prior to opening can be included, thereby providing for extra agitation to the fluid expelled from the reservoir and providing for a higher likelihood that more fluid will be absorbed by the wipe prior to opening the package.

In a specific use example, a person may be using a public lavatory and want to thoroughly cleanse themselves after urinating. The person removes a package from their pocket or purse, the package including one single-use wipe as described herein. The package includes a folded, nonwoven fabric wipe as well as a burst pod including a mild cleanser. The package can also include wording, a graphic, or other similar indicia indicating where the burst pod is located and where the person is to apply pressure in order to rupture the burst pod. The person ruptures the burst pod by applying adequate force to the burst pod through the packaging, thereby dispersing the fluid contained in the burst pod onto the wipe. The person can then open the package using a similar liftable flap as that shown in FIGS. 6A-6B. Upon lifting the liftable flap, the person places one or more fingers into a now-exposed pocket of the wipe and remove the wipe from the package. As the used burst pod is securely adhered to a portion of the packaging, or integrated into the package itself, the burst pod remains inside the package. The person unfolds the wipe and uses the wipe to perform a personal hygiene act. Upon completion of the cleansing process, the person can flush the used wipe in the toilet and throw the package, with the ruptured burst pod, into the garbage.

The above described details and examples are presented for example purposes only. It should be noted that specifics such as materials used, shapes, sizes, fluid quantities, opening mechanism, and other details can be altered based upon the design and implementation of the wipes, burst pods, and packages being used. For example, in certain designs, the burst pod and the packaging can be made from a similar, non-absorbent material such that all fluid released from the burst pod is absorbed by the nonwoven wipe. However, in other designs, the burst pod and the packaging can be made from different materials. For example, the package can be made from a semi-permeable material such that air can pass through the package, while the burst pod is made from an impermeable film to contain the fluid stored therein until the burst pod is ruptured.

As noted above, the wipes as described herein can be saturated with a specific composition (e.g., by a fluid expelled from a ruptured burst pod) to aid a diabetic women in wiping after urination to remove any residual sugar that may be present on or around their vaginal tissue, thereby reducing the chance of a yeast infection. Organisms that colonize humans (whether for a short period [transient] or permanently) do not disrupt normal body functions. In contrast, disease occurs when the interaction between microbe and human leads to a pathologic process characterized by damage to the human host. This process can result from microbial factors (e.g., damage to organs caused by the proliferation of the microbe or the production of toxins or cytotoxic enzymes) or the host's immune response to the organism. While a few infections are caused by strict pathogens (i.e., organisms always associated with human disease such as Mycobacterium tuberculosis (tuberculosis), Neisseria gonorrhoeae (gonorrhea), Francisella tularensis (tularemia), Plasmodium spp. (malaria), and rabies virus (rabies)). Most infections are caused by opportunistic pathogens, organisms that are typically members of the patient's normal microbial flora (e.g., Staphylococcus aureus, Escherichia coli, Candida albicans). These organisms do not produce disease in their normal setting but establish disease when they are introduced into unprotected sites.

The commensal population of the urethra consists of a variety of organisms, with Lactobacilli, Streptococci, and coagulase negative Staphylococci the most numerous. These organisms are relatively avirulent. In contrast, the urethra can be colonized transiently with fecal organisms such as Enterococcus, Enterobacteriaceae, and Candida, all of which can invade the urinary tract, multiply in urine, and lead to significant disease. Pathogens such as N. gonorrhoeae and C. trachomatis are common causes of urethritis and can persist as asymptomatic colonizers of the urethra.

The microbial population of the vagina is diverse and is dramatically influenced by hormonal factors. Newborn girls are colonized with lactobacilli at birth, and these bacteria predominate until the levels of maternal estrogen have declined, and the vaginal flora changes to include anaerobic species such as Staphylococci, Streptococci, and Enterobacteriaceae. When estrogen production is initiated at puberty, the microbial flora changes again. Lactobacilli (especially L. crispatus) re-emerge as the predominant organisms, and many other organisms are also isolated, including Staphylococci (S. aureus less commonly than the coagulase negative species), Streptococci (including group B Streptococcus), Enterococcus, Gardnerella, Mycoplasma, Ureaplasma, Enterobacteriaceae, and a variety of anaerobic bacteria. Vaginosis cases can occur when the balance of vaginal bacteria is disrupted, resulting in decreases in the number of Lactobacilli and increases in the number of Mobiluncus and Gardnerella. Vaginitis and balanitis can occur when C. albicans, Candida glabrata, Candida tropicalis and other fungi proliferate.

As such, vaginal commensal bacteria spp. include: Actinomyces, Bacteroides, Bifidobacterium, Clostridium, Corynebacterium, Enterococcus, Enterobacteriaceae; Eubacterium, Fusobacterium, Gardnerella, Haemophilus, Lactobacillus, Mobiluncus, Mycoplasma, Peptostreptococcus, Propionibacterium, Porphyromonas, Prevotella, Staphylococcus, Streptococcus, Treponema and Ureaplasma, among others.

Gram positive bacteria (e.g., coagulase negative Staphylococcus and less commonly, S. aureus, Corynebacteria, and Propionibacteria) are the most common organisms found on the skin surface. Clostridium perfringens is isolated on the skin of approximately 20% of healthy individuals, and the fungi Candida and Malassezia are also found on skin surfaces, particularly in moist sites. Streptococci can colonize the skin transiently, but the volatile fatty acids produced by the anaerobe Propionibacteria are toxic for these organisms. Gram negative bacilli do not permanently colonize the skin surface (with the exception of Acinetobacter and a few other less common genera) because the skin is too dry. The most common bacteria colonizing the skin comprise Acinetobacter, Aerococcus, Bacillus, Clostridium, Corynebacterium, Micrococcus, Peptostreptococcus, Propionibacterium, Staphylococcus, and Streptococcus.

In the large intestine, more microbes are present than anywhere else in the human body. It is estimated that more than 10¹² bacteria per gram of feces can be found, with anaerobic bacteria in excess by more than 1000 fold. Various yeasts and nonpathogenic parasites can also establish residence in the large intestine. The most common bacteria include Bifidobacterium, Eubacterium, Bacteroides, Enterococcus, and the Enterobacteriaceae. E. coli is present in virtually all humans from birth until death. Although this organism represents less than 1% of the intestinal population, it is the most common aerobic organism responsible for intra-abdominal infections. Likewise, Bacteroides fragilis is a minor member of the intestinal flora but the most common anaerobe responsible for intra-abdominal disease. In contrast, Eubacterium and Bifidobacterium are the most common bacteria in the large intestine, but are rarely responsible for disease.

Antibiotic treatment can rapidly alter the population, causing the proliferation of antibiotic-resistant organisms such as enterococci, Pseudomonas, and fungi. C. difficile can also grow rapidly in this situation, leading to disease ranging from diarrhea to pseudomembranous colitis. Exposure to other enteric pathogens, such as Shigella, enterohemorrhagic E. coli, and Entamoeba histolytica, can also disrupt the colonic flora and produce significant intestinal disease. The most common bacteria colonizing the gastrointestinal tract are Acinetobacter, Actinomyces, Bacteroides, Bifidobacterium, Campylobacter, Clostridium, Corynebacterium, Eubacterium, Enterobacteriaceae, Enterococcus, Fusobacterium, Propionibacterium, Haemophilus, Helicobacter, Lactobacillus, Mobiluncus, Peptostreptococcus, Porphyromonas, Prevotella, Pseudomonas, Staphylococcus, and Streptococcus.

The activity contemplated by the present methods includes both medical therapeutic and/or prophylactic treatment, as appropriate. The specific dose of a cleansing composition administered according to this invention to obtain therapeutic and/or prophylactic effects will, of course, be determined by the particular circumstances surrounding the case, including, for example, the cleansing composition administered, the route of administration, the physical characteristics of the patient (gender, etc.), and the condition being treated. It will be understood that, where necessary, the effective amount administered will be determined by the physician in light of the relevant circumstances, including the condition to be treated, the choice of composition to be administered, and the chosen route of administration, and therefore, the dosage ranges provided are not intended to limit the scope of the invention in any way. A “therapeutically effective amount” of compound of this invention is typically an amount such that when it is administered in a physiologically tolerable excipient composition, it is sufficient to achieve an effective concentration at or in the tissue.

As defined herein an “antibiotic” includes compounds that kill or inhibit the growth of bacteria. An “antimycotic” is defined herein as a compound that kills or inhibits the growth of fungi. A “microbicide” is defined as a compound capable of killing or inhibiting the growth of both bacteria and fungi while optionally being able to inhibit the growth of viruses in certain embodiments.

As disclosed herein, In embodiments, where treatment is desired, the treatment can be achieved by delivering a therapeutic dosage to kill or prevent the growth of a pathogen, but not so as to kill or prevent the growth of at least one commensal species of microflora such as Lactobacillus spp. as in the vagina. Consequently, the concentration of a microbicide, antibiotic, antimycotic, or a combination thereof, to be delivered to a patient may be significantly less than the concentration necessary to sterilize the site of administration. In certain embodiments, the dosage window balancing the anti-pathogenic effects versus the pro-biotic effects is termed “low dose” treatment and comprises a dosage creating a concentration of microbicide, antibiotic, antimycotic, or a combination thereof of 0.5-10 μM locally. Such local concentrations can be achieved by any means known in the art including topical administration.

Those of skill in the art recognize that the dosage of microbicide, antibiotic, antimycotic, or a combination thereof, can be defined in functional terms by titrating the microbicide, antibiotic, antimycotic, or a combination thereof, in a culture. In certain embodiments, such titrations are expressed in terms of minimal inhibitory concentrations “MIC”, which is the lowest antimicrobial concentration that completely inhibits visible growth. Such titrations can be performed in any manner, including using dilution, microdilution, disk diffusion (MIC v. mm area of inhibition), et alia.

In order to preserve the commensal flora at a site of administration, but simultaneously kill or otherwise inhibit a pathogen at the same site, a range of concentrations of a topically administered cleansing compositions may be used. In certain embodiments the range is below the compounds contained in the cleansing compositions' minimal inhibitory concentration of the commensal of interest, such as Lactobacillus crispatus in the vagina; but the concentration is also sufficient to kill or inhibit a pathogen such as C. albicans, especially as it exists in the invasive hyphal form in vaginal candiasis. As such, the concentration necessary to balance growth of a commensal and inhibition of a pathogen at a tissue site simultaneously is expressed in terms of relative effect of the compound on both a pathogen and a commensal or combination of commensals. Thus, in certain embodiments, the concentration of microbicide, antibiotic, antimycotic, or a combination thereof, to be effective against a pathogen is above the minimum inhibitory concentration of the compound on the pathogen, while the same concentration of the microbicide, antibiotic, antimycotic, or a combination thereof, is below the minimum inhibitory concentration so as to spare beneficial species growing at the site of administration.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes eliciting a clinically significant response without excessive levels of side effects.

As such, it is also contemplated that patients are treated for the primary disease in conjunction with the co-morbidity of disease sequelae. In situations where a co-morbidity is associated with susceptibility of infection such as but not limited to susceptibility to vaginitis and balanitis by diabetic patients, treatment of the underlying disease while also treating patients with the disclosed compositions and methods is specifically contemplated. For example in diabetics, a patient can have normal blood glucose, elevated blood glucose, or diminished blood glucose but still derive therapeutic effects through use of the disclosed compositions and methods independent of whether the patient is hyperglycemic, hypoglycemic, or normo-glycemic. Thus, it is contemplated that any patient would benefit from treatment with the disclosed compositions and methods, especially when a patient presents with recurrent infections even where a co-morbidity is not diagnosed. Such embodiments for diabetic treatment are not limited to vaginitis and balanitis infections but also include susceptibility to skin ulceration, including decubitus ulcers.

Thus, a patient will benefit from the disclosed embodiments treating infection while also being treated for diabetes with, for example, sulfonylureas, meglitinides, biguanides, thiazolidinediones, alpha-glucosidase inhibitors, sodium-glucose co-transporter-2 (SGLT2) inhibitors, and dipeptidyl peptidase-4 (DPP-4 inhibitors), or combinations thereof. It is specifically contemplated that diabetic patients treated with second and third generation diabetes drugs would benefit from use of the disclosed embodiments. For example, the embodiments disclosed herein may be used to treat, prevent, mitigate or cure yeast infections associated with diabetes treatment with DPP-4 inhibitors, (e.g., itagliptin, vildagliptin, saxagliptin, linagliptin, dutogliptin, gemigliptin, alogliptin, berberine, etc.), as well as SGLT2 inhibitors (e.g., canagliflozin, dapagliflozin, ipragliflozin, empagliflozin, tofogliflozin, etc.). In yet other embodiments, a patient can be treated with two or more drugs for the underlying diabetic condition, while still benefitting from the compositions and methods disclosed.

In yet other embodiments, diabetic patients being treated with drugs such as, but not limited to, sulfonylureas, meglitinides, biguanides, thiazolidinediones, alpha-glucosidase inhibitors, sodium-glucose co-transporter-2 (SGLT2) inhibitors, and dipeptidyl peptidase-4 (DPP-4 inhibitors) may experience glycosuria during the course of treatment. Glycosuria is appreciated as a potential risk for urinary tract and genital infections such as but not limited to vulvovaginal candidiasis infections. Without wishing to be bound by theory, the presence of excess glucose in the urine may create a microenvironment that promotes the colonization of the urinary tract and genital area with pathogenic microorganisms such as but not limited to Candida albicans and other microbes leading to painful and chronic episodes of vaginitis, balanitis, and skin infections. Accordingly, compositions capable of sequestering glucose may be useful in reducing a patient's susceptibility to infection. In yet other embodiments, these compositions may additionally comprise one or more microbicidal agent capable of killing pathogenic microrganisms. In some embodiments, the compositions and methods disclosed herein may reduce the risk of urinary tract and genital infections in a patient with glycosuria. In some embodiments, the compositions promote the growth and health of endogenous microflora.

In some embodiments, the compositions described herein may have a cleansing function. As used herein, the term “cleansing” as used herein refers to the process of removing substances from the surface of the skin including but not limited to dirt, urine, faeces, oil, and glucose. In some embodiments, the cleansing function of the compositions described herein is achieved through the use of an article comprising a cleansing composition incorporated into a substrate.

### Cleansing Compositions

Embodiments, herein are directed to compositions including but not limited to cleansing compositions. These compositions may be incorporated into a substrate such as the nonwoven wipes as described above or administered directly. The cleansing compositions of the present embodiments are useful for reducing the risk of urinary tract and genital infections resulting from glycosuria resulting from, for example, diabetes.

In certain implementations, the cleansing composition as described herein includes glucose oxidase. Glucose oxidase, also known as notatin, is an oxido-reductase enzyme that catalyzes the oxidation of glucose to hydrogen peroxide and glucono delta-lactone. Glucose oxidase also displays antibacterial activity when exposed to oxygen and glucose as a result of producing hydrogen peroxide.

In some embodiments, the cleansing composition may comprise a microbicide, antibiotic, antimycotic, or a combination thereof. In some embodiments, the microbicide, antibiotic, antimycotic, or a combination thereof, may include but are not limited to small molecules, nucleic acids, proteins, peptides, or combinations thereof. In some embodiments, certain compositions are specifically contemplated. For example, in some embodiments, compositions contemplated for treating, preventing or reducing the risk of bacterial infections in a patient comprise but are not limited to bacitracin, bacitracin/polymyxin B, bacitracin/neomycin/polymyxin B, neomycin/polymyxin B/hydrocortisone, bacitracin/neomycin/polymyxin B/hydrocortisone, benzalkonium chloride, benzalkonium chloride/pramoxine, benzalkonium chloride/lidocaine, benzethonium, benzethonium chloride/benzocaine, benzethonium chloride/lidocaine, borax, chlorhexidine, clindamycin, gentamicin, mupirocin, mafenide, phenol, retapamulin, silver sulfadiazine, sulfacetamide, sulfanilamide or any combination thereof.

In some embodiments, compositions contemplated for treating, preventing or reducing the of risk fungal infections in a patient may comprise but are not limited to borate salt, butenafine, butoconazole, ciclopirox, clotrimazole, clotrimazole/betamethasone dipropionate, econazole, gentian violet, ketoconazole, naftifine, nystatin, nystatin/triamcinolone, oxiconazole, quaternary ammonium salts, silane quaternary ammonium salts such as 3-(trihydroxysilyl) propyldimethyloctadecyl ammonium chloride, selenium sulfide, pyrithione zinc, miconazole, miconazole/zinc oxide/petrolatum, tolnaftate, terbinafine, tioconazole, sertaconazole, sulconazole, terbinafine and combinations thereof.

In some embodiments, the compositions described herein may comprise an antimycotic agent such as but not limited to the imidazole class of antifungal agents including, for example, but not limited to butoconazole, clotrimazole, econazole, ketoconazole, miconazole, oxiconazole, sertaconazole, sulconazole, tioconazole, and combinations thereof.

In some embodiments, the local concentration of microbicide, antibiotic, antimycotic, or a combination thereof, in the cleansing composition is about 0.5-10 μM/UL. Those of skill in the art recognize that such a concentration is easily convertible among equivalents. For example, where the molecular weight of a compound is 1000 MW, the solute mass in a 1 mg/L solution is 1 mM. Similarly, the use of the volume in the denominator is not necessary to describe the molarity of a solution. Therefore, as in the above example, a 1 mM solution would comprise a ratio of 1 μg/ml of water.

In some embodiments, the concentrations of a microbicide, antibiotic, antimycotic, or a combination thereof, able to kill or inhibit the growth of a pathogen can be about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10.0 μM, 20 μM, 30 μM, 40 μM, 50 μM, 60 μM, 70 μM, 80 μM, 90 μM, 100 μM, or higher. It is also contemplated that a microbicide, antibiotic, antimycotic, or a combination thereof, can be delivered locally to a site such as a mucosa or skin so that the concentration in situ is about 0.5 μM-10 μM.

In some embodiments, the cleansing compositions may comprise agents that regulate the concentration of nutrients at the site of administration, such as mineral sequestration agents (e.g., siderophiles, chelation agents, etc.), glucose sequestration agents, nitrogen sequestration agents, phosphorous sequestration agents, and other compounds that can regulate the composition and concentration of necessary nutrients and minerals at the site of application. In some embodiments, glucose sequestration agents include but are not limited to a zeolite, aluminum oxide microspheres, ceramic microspheres, hydrous alumina silicate microspheres, alumina dessicant beads, attapulgus clay, beaded silica gel desiccants, natural clay absorbents, activated carbon or any combination thereof. In some embodiments, glucose sequestration agents include but are not limited to molecular sieve ABSCENTS and MOLSIV GMP brand of synthetic or natural zeolite based deodorizing powders of highly controlled pore size with internal pore sizes up to 700 M2/tablespoon (UOP International, Des Plaines, Ill.), Versal synthetic aluminum oxide microspheres A 1203, A-201, and A-2 as absorbents (UOP International); synthetic ceramic microspheres as inert absorbents, Zeospheres brand (Lawrence Industries, Ltd, UK), ASP Series hydrous alumina silicate microspheres as absorbents (Lawrence Industries); Dryocel alumina desiccant beads with high surface area (up to 400 M2/gm internal surface area) as high capacity absorbents (Lawrence Industries); Pharmasorb attapulgus clay with high absorptivity at select pore size (Lawrence Industries); Trockenperlon beaded silica gel desiccants as absorbents (Lawrence Industries); natural clay absorbents such as chabazite mineral zeolite ZS500H, ZS500a, ZS500RW, ZS500AA, or A or the like (GSA Resources, Inc., Tucson, Ariz.); additional natural clay absorbents such as: clay Ferrierite CP914; ZSM-5 Type Zeolite CBV 3024E, 5534G, 8014, or 28014; Zeolite Y Type CBV100-901, Clinoptilolite; Mordenite type CBV 10A, 21A, or 90A (Zeolyst International, Valley forge, Pa.); activated carbon; or any combination thereof.

In yet other embodiments, the cleansing compositions can include commensal specific growth enhancing agents such as non-nitrosyl sugars and other compounds preferentially encouraging outgrowth of commensal microflora but not pathogenic microflora. In some other embodiments, the cleansing compositions comprise pro-biotic cultures of commensal microflora for helping reestablish the normal population of microbiota at the site of administration. In yet other embodiments, the cleansing compositions can also comprise topical analgesics. In some other embodiments, the compositions can comprise anti-pruritic agents.

In some embodiments, the cleansing compositions described herein may comprise one or more cleansing agents. In some embodiments, the cleansing compositions may be aqueous cleansing compositions. In some embodiments, the cleansing compositions comprise one or more cleansing surfactants. In some embodiments, the cleansing compositions comprise an effective amount of one or more cleansing surfactants. In some embodiments the effective amount of one or more cleansing surfactant provides a cleansing benefit. In some embodiments, without wishing to be bound by theory, surfactants or combinations of surfactants should be mild, which means that these surfactants provide sufficient cleansing or detersive benefits but do not overly dry the skin. In some embodiments, cleansing surfactants include but are not limited to anionic surfactants, nonionic surfactants, amphoteric surfactants, cationic surfactants, and combinations thereof. In some embodiments, anionic surfactants, nonionic surfactants, amphoteric surfactants, cationic surfactants, and combinations thereof include but are not limited to those disclosed in McCutcheon's, Detergents and Emulsifiers. North American edition (2013); McCutcheon's, Functional Materials. North American Edition (2013) each of which are incorporated by reference herein in their entirety. In some embodiments, anionic surfactants include but are not limited to sarcosinates, sulfates, isethionates, taurates, phosphates, and combinations thereof. In some embodiments, isethionates include but are not limited to alkoyl isethionates, alkyl sulfates and alkyl ether sulfates. In some embodiments, alkoyl isethionates include but are not limited to ammonium cocoyl isethionate, sodium cocoyl isethionate, sodium lauroyl isethionate, and combinations thereof. In some embodiments, synthetic surfactants include but are not limited to succinamates, olefin sulfonates, b-alkyloxy alkane sulfonates and combinations thereof. Examples of synthetic surfactants include but are not limited to sodium lauryl sulfate and ammonium lauryl sulfate. In some embodiments, additional anionic materials include but are not limited to sarcosinates, such as but not limited to sodium lauroyl sarcosinate, sodium cocoyl sarcosinate, and ammonium lauroyl sarcosinate. In yet other embodiments, anionic materials may include but are not limited to soaps including but not limited to alkali metal salts, such as but not limited to, sodium or potassium salts. Some embodiments may include soaps derived from fatty acids. The fatty acids used in making these soaps can be obtained from natural sources such as, for instance, plant or animal-derived glycerides (e.g., palm oil, coconut oil, soybean oil, castor oil, tallow, lard, etc.) The fatty acids can also be synthetically prepared. In some embodiments, anionic materials include phosphates such as but not limited to monoalkyl, dialkyl, and trialkylphosphate salt, alkanoyl sarcosinates, such as but not limited to, lauroyl sarcosinate, taurates such as but not limited to N-alkyltaurines. In some embodiments, anionic surfactants include but are not limited to, sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl sarcosinate, and combinations thereof.

In some embodiments, nonionic surfactants for use in the cleansing compositions described herein include but are not limited to polyoxyethylenes, alkyl glucosides, alkyl polyglucosides, polyhydroxy fatty acid amides, alkoxylated fatty acid esters, sucrose esters, amine oxides, alkyl glucosides and alkyl polyglucosides sucrose cocoate and sucrose laurate, polyhydroxy fatty acid amide surfactants, coconut alkyl N-methyl glucoside amide, amine oxides, polyoxyethylenes, C8-C14 glucosamides, C8-C14 alkyl polyglucosides, sucrose cocoate, sucrose laurate, lauramine oxide, cocoamine oxide and combinations thereof. Commercially available examples of nonionic surfactants include decyl polyglucoside (available as APG 325 CS from Henkel) and lauryl polyglucoside (available as APG 600CS and 625 CS from Henkel). In some embodiments, examples of amine oxides include but are not limited to dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, and dimethylhexadecylamine oxide.

The term “amphoteric surfactant,” as used herein, is also intended to encompass zwitterionic surfactants, which are well known to formulators skilled in the art as a subset of amphoteric surfactants. A wide variety of amphoteric surfactants can be used in the compositions described herein. In some embodiments, amphoteric surfactants include but are not limited to derivatives of aliphatic secondary and tertiary amines, wherein in some embodiments, the nitrogen is in a cationic state, in which the aliphatic radicals can be straight or branched chain and wherein one of the radicals contains an ionizable water solubilizing group, e.g., carboxy, sulfonate, sulfate, phosphate, or phosphonates. In some embodiments, examples of amphoteric or zwitterionic surfactants include but are not limited to betaines, sultaines, hydroxysultaines, alkyliminoacetates, iminodialkanoates, aminoalkanoates, amphoteric phosphates, ammonium derivatives and combinations thereof. In some embodiments, examples of betaines include but are not limited to higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl) sulfopropyl betaine, amidobetaines and amidosulfobetaines, oleyl betaine, cocamidopropylbetaine, sodium 3-dodecyl-aminopropionate, sodium 3-dodecylaminopropane sulfonate, and cocamidopropyl betaine. In some embodiments, examples of sultaines and hydroxysultaines include but are not limited to cocamidopropyl hydroxysultaine.

In some embodiments, examples of cationic surfactants include but are not limited to cationic alkyl ammonium salts, amino-amides and quatrernary ammonium salts. In some embodiments, examples of cationic alkyl ammonium salts, amino-amides and quatrernary ammonium salts include but are not limited to stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and combinations thereof. In some embodiments, quaternary ammonium salts also include but are not limited to those wherein the C12 to C22 alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C16 to C18 range. The term “coconut” refers to an alkyl group derived from a coconut fatty acid, which generally have mixtures of alkyl chains in the C 12 to C14 range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and combinations thereof. In some embodiments, cationic surfactants may also include dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, and combinations thereof.

In some embodiments, the cleansing compositions described herein may also comprise conditioning agents. In some embodiments, conditioning agents include but are not limited to lipophilic skin conditioning agents which are useful for providing a conditioning benefit to the skin.

The conditioning agents described herein may comprise from about 0.1% to about 30%, from about 0.5% to about 20%, from about 1% to about 10%, or from about 1% to about 5% by weight of compositions described herein.

In some embodiments, examples of lipophilic skin conditioning agents include but are not limited to mineral oil, petrolatum, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cylcomethicones having 3 to 9 silicon atoms, vegetable oils, hydrogenated vegetable oils, polypropylene glycol C4-C20 alkyl ethers, di C8-C30 alkyl ethers, dodecane, isododecane, squalane, cholesterol, hydrogenated polyisobutylene, docosane, hexadecane, isohexadecane, C7-C40 isoparaffins, C1-C30 alcohol esters of C1-C30 carboxylic acids and of C2-C30 dicarboxylic acids, including but not limited to straight and branched chain materials as well as aromatic derivatives, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, straight chain, branched chain and aryl carboxylic acids, C1-C30 monoesters and polyesters of sugars, solid and liquid esters, propoxylated and ethoxylated derivatives of these materials and combinations thereof. In some embodiments, lipophilic skin conditioning agents include but are not limited to diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprilic/capric triglyceride, PEG-6 caprylic/capric triglyceride, PEG-8 caprylic/capric triglyceride, and combinations thereof.

In some embodiments, the cleansing compositions described herein may also comprise a secondary active ingredient that can be categorized by its therapeutic benefit or postulated mode of action. However, it is to be understood that the active ingredients useful herein can in some instances provide more than one therapeutic benefit or operate via more than one mode of action. Therefore, classifications herein are made for the sake of convenience and are not intended to limit the active ingredient to that particular application or applications listed. Also, pharmaceutically-acceptable salts of these active ingredients are useful herein.

In some embodiments, the secondary active ingredient is a non-steroidal anti-inflammatory agent such as, but not limited to, propionic acid derivatives, acetic acid derivatives, fenamic acid derivatives, biphenylcarboxylic acid derivatives, oxicams, acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, diclofenac, etodolac, fluprofen and bucloxic acid, hydrocortisone and combinations thereof.

In some embodiments, the secondary active ingredient is a topical anesthetics such as, but not limited to, benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

In some embodiments, the secondary active ingredient is an antimicrobial and antifungal agent such as but not limited to β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole and combinations thereof.

In some embodiments, secondary active ingredients may include benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, neocycin sulfate, salicylic acid, niacinamide, cis-retinoic acid, trans-retinoic acid, retinol, retinyl palmitate, phytic acid, N-acetyl L-cysteine, azelaic acid, lipoic acid, resorcinol, lactic acid, glycolic acid, ibuprofen, naproxen, hydrocortisone, phenoxyethanol, phenoxypropanol, phenoxyisopropanol, 2,4,4, ′-trichloro-2′-hydroxy diphenyl ether, 3,4,4′-trichlorocarbanilide, 2-ethylhexyl-methoxycinnamic acid, oxybenzone, 2-phenylbenzimidozole-5-sulfonic acid, dihydroxyacetone, and combinations thereof.

In some embodiments, the cleansing compositions disclosed herein may further comprise a proton donating agent. By “proton donating agent” it is meant any acid compound or mixture thereof, which results in undissociated acid on the skin after use. Proton donating agents can be organic acids, including polymeric acids, mineral acids or mixtures thereof.

In some embodiments, examples of organic acids which can be used as the proton donating agent include but are not limited to adipic acid, tartaric acid, citric acid, maleic acid, malic acid, succinic acid, glycolic acid, glutaric acid, benzoic acid, malonic acid, salicylic acid, gluconic acid, polymeric acids, their salts, and mixtures thereof. A non-exclusive list of examples of mineral acid for use herein are hydrochloric, phosphoric, sulfuric and combinations thereof. In some embodiments, the cleansing compositions described herein may comprise a polymeric acid. Polymeric acids are especially preferred acids for use herein from the standpoint that they cause less stinging to the skin than other acids. As used herein, the term “polymeric acid” refers to an acid with repeating units of carboxylic acid groups joined together into one chain. In some embodiments, examples of polymeric acids include homopolymers, copolymers and terpolymers, straight-chain poly(acrylic) acid and its copolymers, both ionic and nonionic, (e.g., maleic-acrylic, sulfonic-acrylic, and styrene-acrylic copolymers), those cross-linked polyacrylic acids having a molecular weight of less than about 250,000, preferably less than about 100,000 poly (α-hydroxy) acids, poly (methacrylic) acid, naturally occurring polymeric acids such as carageenic acid, carboxy methyl cellulose, alginic acid, straight-chain poly (acrylic) acids and combinations thereof. In some embodiments, the proton donating agent is used to buffer the pH of the aqueous cleansing composition to a pH ranging from about 3.0 to about 6.0, more preferably from about 3.0 to about 5.0 and most preferably from about 3.5 to about 4.5.

In some embodiments, the cleansing compositions described herein may also comprise water soluble conditioning agents. In some embodiments, water soluble conditioning agents may be present in the compositions described herein at a level ranging from about 0.1% to about 2%, from about 0.2% to about 1.5%, from about 0.5% to about 1% by weight of the composition. In some embodiments, water soluble conditioning agents include but are not limited to polyhydric alcohols, polypropylene glycols, polyethylene glycols, ureas, pyrrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, urea; guanidine; glycolic acid and glycolate salts (e.g., ammonium and quaternary alkyl ammonium); lactic acid and lactate salts (e.g., ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-30, PEG-polypropylene glycols such as PPG-9, PPG-12, PPG-15, PPG-17, PPG-20, PPG-26, PPG-30, PPG-34; alkoxylated glucose; hyaluronic acid; and combinations thereof.

In some embodiments, the cleansing compositions described herein may also comprise materials such as aloe vera in any of its variety of forms (e.g., aloe vera gel), chitin, starch-grafted sodium polyacrylates such as Sanwet® IM-1000, IM-1500, and IM-2500 (available from Celanese Superabsorbent Materials, Portsmouth, VA); lactamide monoethanolamine; acetamide monoethanolamine; and combinations thereof.

In some embodiments, the cleansing compositions described herein may comprise drying agents. In some embodiments, drying agents may accelerate the drying rate of a composition once it is applied to the skin. In some embodiments, drying agents include isoparaffin, alcohols and combinations thereof. In some embodiments, drying agents may be present in the compositions described herein at a level ranging from about 1% to about 60%, from about 3% to about 40%, or from about 5% to about 20% by weight.

In some embodiments, the cleansing compositions described herein may contain additives such as odor-control additives, preservatives, or moisturizing additives. In some embodiments, these additives will not impair or reduce the therapeutic effectiveness or reduce the benefit of the composition.

The cleansing compositions described herein can comprise a wide range of other optional components. These additional components should be pharmaceutically acceptable. The CTFA Cosmetic Ingredient Handbook, Twelfth Edition, 2008, which is incorporated by reference herein in its entirety, describes a wide variety of cosmetic and pharmaceutical ingredients commonly used in the skin care industry, which may be suitable for use in the compositions described herein. Examples of these and other functional classes include, but are not limited to abrasives, absorbents, anticaking agents, antioxidants, vitamins, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, preservatives, propellants, reducing agents, skin bleaching agents, fragrances, pigments, colorings, essential oils, skin sensates, astringents, skin soothing agents, and skin healing agents.

In some embodiments, the cleansing compositions may comprise a pharmaceutically acceptable carrier, diluent, or excipient to preserve, stabilize, or otherwise enhance the effects of the compositions. The compositions of the present embodiments may be formulated in any way. For example, in various embodiments, the compositions may be formulated as a liquid, an aerosol, solid, gel, lotion, or cream, or may be embedded into a substrate and the formulation of the composition may vary among embodiments depending on the mode of administration of the compositions.

In some embodiments, the cleansing compositions described herein can be topical dosage forms including, but not limited to, solutions, sprays, aerosols, powders, fluid emulsions, fluid suspensions, semi-solids, ointments, pastes, creams, lotions, gels, jellies, and foams; and parenteral dosage forms including, but not limited to, solutions, suspensions, emulsions, and dry powders. The compositions described herein can also be formulated in rectally or vaginally administered compositions, such as, suppositories or retention enemas, for example, containing conventional suppository bases such as cocoa butter or other glycerides. The active ingredients can be contained in such compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, and the like.

In some embodiments, the cleansing compositions described herein also can include suitable solid or gel phase carriers or excipients. Examples of such carriers or excipients include but are not limited to calcium carbonate, calcium phosphate, gelatin, and polymers such as, for example, polyethylene glycols.

In some embodiments, the cleansing compositions described herein can also be administered in combination with other active ingredients, such as, for example, adjuvants, protease inhibitors, or other compatible drugs or compounds where such combination is seen to be desirable or advantageous in achieving the desired effects of the methods described herein.

The means and methods for administration of the cleansing compositions described herein are known in the art and an artisan can refer to various pharmacologic references, such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co., New York (1980) for guidance.

For administration by aerosol, the cleansing compositions described herein may be delivered in the form of an aerosol spray presentation with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount.

The cleansing compositions described in the embodiments above may be administered to any tissue having an infection or at risk of infection. For example, in some embodiments, such compositions may be administered to vaginal mucosa, vulva, labia, perineum, skin or skin folds, foreskin, pubic areas, hair, oral or nasal mucosa, gastrointestinal mucosa, and transition zones between mucosa and skin including perianally, periurethrally, and periumbically. For prevention of infections during breast feeding, the compositions may be used on the nipple and areola.

The amount of the cleansing compositions of various embodiments to be administered is an amount that is therapeutically effective, and the dosage administered may depend on the characteristics of the subject being treated. For example, the dosage may depend on the particular animal treated, the age, weight, and health of the subject, the types of concurrent treatment, if any, and frequency of treatments. Many of these factors can be easily determined by one of skill in the art (e.g., by the clinician).

Some embodiments are directed to a pharmaceutical composition comprising: a microbicide at a concentration sufficient to kill at least one pathogenic microorganism and at a concentration below the minimal inhibitory concentration for growth of at least one commensal organism; and a pharmaceutically acceptable excipient.

### Substrates

In some embodiments, the cleansing compositions may be incorporated into a substrate such as the nonwoven clothes as described above. In some embodiments, the cleansing compositions described herein may be incorporated into a substrate such as a wipe. As used herein the terms “wipe” and “towlette” are to be used interchangeably. The fibrous substrate may be, for example, a non-woven fabric or a woven fabric such as for example, wipes and towelettes. In some examples, the cleaning composition can be included in a frangible reservoir or similar burst pod and packaged in a disposable contained with a dry wipe. Prior to opening the container, the burst pod can be ruptured, thereby releasing the cleaning composition to saturate the wipe.

As noted above, non-woven fabric may be made from a variety of processes, for example, hydroentangling technique, air-laid process, wet-laid process, solution spinning or chemical bonding. In some embodiments, the substrate can be formed from natural fibers, synthetic fibers or combinations thereof. Suitable natural fibers from which to prepare the substrates herein include, for example, wood pulp, wool, silk, jute, hemp, cotton, linen, sisal, ramie and combinations thereof. Suitable synthetic fibers from which the substrates herein can be prepared include rayon, cellulose ester, polyvinyl derivatives, polyolefins, polyamides, acetate, acrylic, modacrylic fibers, polyester, polyurethane foam, and combinations thereof. Specific examples of some of these synthetic materials include acrylics such as acrilan, creslan, and the acrylonitrile-based fiber, orlon; cellulose ester fibers such as cellulose acetate, arnel, and acele; polyamides such as nylons (e.g., nylon 6, nylon 66, nylon 610, and the like); polyesters such as fortrel, kodel, and the polyethylene terephthalate fiber, dacron; polyolefins such as polypropylene, polyethylene; polyvinyl acetate fibers; polyurethane foams and combinations thereof. In some embodiments the fibrous substrate may comprise paper tissue, toilet paper, cellulose, polyvinyl alcohol, guar gum, starch, a polymer, or combinations thereof.

In some embodiments, the fibrous substrate may be embossed, molded, wetted, or textured to create a specific wipe design. The wipe may be a single layer or a plurality of layers where the layers are generally positioned in a surface-to-surface relationship where all or a portion of the layers may be bound to adjacent layers. In certain embodiments, the wipe is a water-dispersible, flushable wipe product that is stable during storage and use, but upon contact with water will readily disperse. The wipe is water-dispersible in all hard and soft types of water found in toilets throughout the United States and the world.

In some embodiments, the wipe is a wet wipe and additionally comprises a wetting composition to allow for the wet wipe to maintain a desirable level of strength. The wetting composition may provide some additional advantages, such as improved tactile properties, sanitizing properties, a cooling sensation when applied to the skin or mucosa, and combinations thereof. In some embodiments, the wetting composition of the wet wipe may include an insolubilizing content, such as a polyol or a lower level alcohol, glycol, ketone, or any combination thereof. These insolubilizing agents have a tailored sensitivity which maintains integrity or wet strength of the fibrous substrate, but upon contact with water the resulting treated fibrous substrate readily disperses once the polyol or alcohol concentration falls below a critical level. In relation to the weight of the dry fibrous substrate, the wet wipe may contain from about 10 percent to about 500 percent of the wetting composition, 50 percent to about 400 percent of the wetting composition, 100 percent to about 300 percent of the wetting composition, or any percentage between any of these listed values.

The cleansing compositions disclosed herein can be incorporated or absorbed into the wipe, and in relation to the dry fibrous substrate, may be about 0.01 weight percent, about 0.05 weight percent, about 0.1 weight percent, about 0.5 weight percent, about 1 weight percent, about 2 weight percent, about 5 weight percent, about 10 weight percent, or any percentage between any of these listed values.

Cleansing Compositions Incorporated into Substrates

Any method suitable for the application of the cleansing compositions described herein, including flood coating, spray coating or metered dosing, can be used to impregnate the substrates herein with the compositions described herein. In some embodiments, techniques, such as Meyer Rod, floating knife or doctor blade, which are typically used to impregnate cleansing solutions into absorbent sheets may also be used.

In some embodiments, prior to or after impregnation of the cleansing compositions described herein into a substrate, the substrate may be folded into stacks. In some embodiments, the substrate is then typically packaged in a moisture and vapor impermeable package.

The wipe product may be individually packaged or packaged in a container with any number of wipes. In some embodiments, the wipes are in a folded condition and individually wrapped for single use design. In other embodiments, the wipes are in a folded condition and packaged in a container or envelope that is resealable. In other embodiments, the wipes are wet wipes and the container or envelope is moisture-proof and impermeable. In further embodiments, the final wipe product may be packaged as a roll of separable sheets in a moisture-proof container. The roll may contain a hollow or solid core or may be coreless. The final wipe product may retain all desired characteristics over a time period that includes warehousing, transportation, retail display, and storage by the consumer.

Some embodiments are directed to a wipe comprising less than the minimal inhibitory concentration of 3-(trihydroxysilyl) propyldimethyloctadecyl ammonium chloride for growth of at least one commensal organism.

In yet other embodiments, the cleansing compositions described herein may be incorporated into a panty liner comprising a porous inner layer intended to face the skin, an absorbent middle layer and a nonporous outer layer intended to face the underwear. In some embodiments, the absorbent middle layer comprises the compositions described herein. In some embodiments, the middle layer comprises one or more sequestration agent. In some embodiments, the absorbent middle layer comprises one or more glucose sequestration agents. In some embodiments, glucose sequestration agents include but are not limited to a zeolite, aluminum oxide microspheres, ceramic microspheres, hydrous alumina silicate microspheres, alumina dessicant beads, attapulgus clay, beaded silica gel desiccants, natural clay absorbents, activated carbon or any combination thereof.

In some embodiments, the compositions described herein may be contained in a panty liner having a body-facing side, a garment facing side, two longitudinal edges and two end edges. In some embodiments, the panty liner comprises a liquid pervious topsheet and a liquid impervious backsheet joined to said topsheet, and an absorbent core positioned between said topsheet and said backsheet. In some embodiments, the absorbent core comprises one of more one or more glucose sequestration agents such as but not limited to a zeolite, aluminum oxide microspheres, ceramic microspheres, hydrous alumina silicate microspheres, alumina dessicant beads, attapulgus clay, beaded silica gel desiccants, natural clay absorbents, activated carbon or any combination thereof. In some embodiments, glucose sequestration agents include but are not limited to a zeolite, aluminum oxide microspheres, ceramic microspheres, hydrous alumina silicate microspheres, alumina dessicant beads, attapulgus clay beaded silica gel desiccants, natural clay absorbents, activated carbon or any combination thereof.

As used herein the term “longitudinal” refers to an imaginary line, axis or direction of the panty liner, which line, axis or direction is typically centered between the side margins of the panty liner and is generally aligned with the vertical plane which bisects a standing wearer into left and right body halves. The terms “lateral” or “transverse” refer to an imaginary line, axis or direction generally orthogonal to the longitudinal direction and within the plane of the panty liner, which is generally sideways aligned relative to the wearer.

Some embodiments are directed to a cleansing article comprising: a substrate; and a cleansing composition; wherein the cleansing composition is incorporated into the substrate. In some embodiments, the substrate is a fibrous substrate. In some embodiments, the fibrous substrate is a non-woven fabric. In some embodiments, the substrate comprises paper tissue, toilet paper, cellulose, polyvinyl alcohol, guar gum, starch, a polymer, or any combinations thereof. In some embodiments, the substrate is a wipe. In some embodiments, the substrate is a panty liner.

In some embodiments, the cleansing composition comprises a wetting composition. In some embodiments, the wetting composition comprises a polyol, a lower level alcohol, a glycol, a ketone, or any combination thereof. In some embodiments, the cleansing composition comprises a sequestering agent. In some embodiments, the sequestering agent is a glucose sequestering agent. In some embodiments, the glucose sequestering agent is selected from a zeolite, aluminum oxide microspheres, ceramic microspheres, hydrous alumina silicate microspheres, alumina dessicant beads, attapulgus clay, beaded silica gel desiccants, natural clay absorbents, activated carbon or any combination thereof. In some embodiments, the cleansing agent is an aqueous cleansing composition.

In some embodiments, the cleansing composition comprises at least one cleansing surfactant. In some embodiments, the surfactant is present in an effective amount to provide a cleansing benefit. In some embodiments, the cleansing surfactant is an anionic surfactant, a nonionic surfactant, an amphoteric surfactant, a cationic surfactant, or any combination thereof.

In some embodiments, the cleansing composition comprises a conditioning agent. In some embodiments, the conditioning agent is a lipophilic conditioning agent.

In some embodiments, the cleansing composition comprises a secondary active ingredient selected from a non-steroidal anti-inflammatory agent, a topical anesthetic, and a combination thereof.

In some embodiments, the cleansing composition comprises an antibiotic agent. In some embodiments, the concentration of the antibiotic agent in the cleansing composition is sufficient to kill at least one pathogenic microorganism and at a concentration below the minimal inhibitory concentration for growth of at least one commensal organism. In some embodiments, the antibiotic agent is selected from β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole and combinations thereof.

In some embodiments, the cleansing composition comprises an microbicidal agent. In some embodiments, the concentration of the microbicidal agent in the cleansing composition is sufficient to kill at least one pathogenic microorganism and at a concentration below the minimal inhibitory concentration for growth of at least one commensal organism. In some embodiments, the microbicidal agent is selected from bacitracin, bacitracin/polymyxin B; bacitracin/neomycin/polymyxin B, neomycin/polymyxin B/hydrocortisone, bacitracin/neomycin/polymyxin B/hydrocortisone, benzalkonium chloride, benzalkonium chloride/pramoxine, benzalkonium chloride/lidocaine, benzethonium, benzethonium chloride/benzocaine, benzethonium chloride/lidocaine, borax, chlorhexidine, clindamycin, gentamicin, mupirocin, mafenide, phenol, retapamulin, silver sulfadiazine, sulfacetamide, sulfanilamide, borax, butenafine, butoconazole, ciclopirox, clotrimazole, clotrimazole/betamethasone dipropionate, econazole, gentian violet, ketoconazole, naftifine, nystatin, nystatin/triamcinolone, oxiconazole, quaternary ammonium salts, silane quaternary ammonium salts, 3-(trihydroxysilyl) propyldimethyloctadecyl ammonium chloride, selenium sulfide, pyrithione zinc, miconazole, miconazole/zinc oxide/petrolatum, tolnaftate, terbinafine, tioconazole, sertaconazole, sulconazole, terbinafine and combinations thereof.

In some embodiments, the cleansing composition comprises an antimycotic agent. In some embodiments, the concentration of the antimycotic agent is the cleansing composition is sufficient to kill at least one pathogenic microorganism and at a concentration below the minimal inhibitory concentration for growth of at least one commensal organism. In some embodiments, the antimycotic agent is selected from borate salt, clotrimazole, oxiconazole, miconazole, ketoconazole, econazole, butoconazole, tioconazole, sertaconazole, sulconazole, and combinations thereof.

In some embodiments the cleansing compositions disclosed herein can be incorporated into a substrate that is then used for the manufacture of an adult diaper. In some embodiments, the cleansing compositions disclosed herein can be incorporated into a substrate and may be useful when used in articles intended to be used in and around or worn over the urogenital area. For example, the cleansing compositions disclosed herein can be incorporated into a substrate that is configured as an adult diaper wherein the wearer's urogenital area is potentially exposed to urine for a prolonged period of time.

In some embodiments, the cleansing compositions disclosed herein can be incorporated into a substrate that is configured into article in a single use packaging that can be stored and used discreetly and disposed of after use. In yet other embodiments, the cleansing compositions disclosed herein can be incorporated into a substrate can be configured into an article that is contained in a multi-item packaging such that the packaging can be opened and reclosed and preserved the integrity of the cleansing compositions disclosed herein and substrates.

Methods of Using Cleansing Compositions Incorporated into Substrates

The cleansing compositions described in the embodiments above may be administered to any tissue having an infection or at risk of infection. For example, in some embodiments, such compositions may be administered to vaginal mucosa, vulva, labia, perineum, skin or skin folds, foreskin, pubic areas, hair, oral or nasal mucosa, gastrointestinal mucosa, and transition zones between mucosa and skin including perianally, periurethrally, and periumbically. For prevention of infections during breast feeding, the compositions may be used on the nipple and areola.

The amount of the cleansing compositions of various embodiments to be administered is an amount that is therapeutically effective, and the dosage administered may depend on the characteristics of the subject being treated. For example, the dosage may depend on the particular animal treated, the age, weight, and health of the subject, the types of concurrent treatment, if any, and frequency of treatments. Many of these factors can be easily determined by one of skill in the art (e.g., by the clinician).

Some embodiments are directed to methods of treating infection at a colonized tissue site, comprising topically administering a microbicide to a tissue surface at a concentration sufficient to kill at least one pathogenic microorganism and at a concentration below the minimal inhibitory concentration for growth of at least one commensal organism. In some embodiments, the tissue surface is selected from the group consisting of skin, mucosa, and transition zones of mucosa. In some embodiments, the transition zone is selected from the group consisting of lips, perianal tissue, periurethral tissue, and periumbical tissue. In some embodiments, the tissue surface is selected from the group consisting of vaginal mucosa, vulva, labia, perineum, skin, skin folds, foreskin, pubic region, nipple, areola, hair, oral, mucosa, nasal mucosa, and gastrointestinal mucosa.

In some embodiments, the mucosa is vaginal mucosa. In some embodiments, the colonizing commensal is Lactobacillus species. In some embodiments, the Lactobacillus species is Lactobacillus crispatus. In some embodiments, the disease is a bacterial infection. In some embodiments, the microbicide is selected from the group consisting of bacitracin, bacitracin/polymyxin B; bacitracin/neomycin/polymyxin B, neomycin/polymyxin B/hydrocortisone, bacitracin/neomycin/polymyxin B/hydrocortisone, benzalkonium chloride, benzalkonium chloride/pramoxine, benzalkonium chloride/lidocaine, benzethonium, benzethonium chloride/benzocaine, benzethonium chloride/lidocaine, borax, chlorhexidine, clindamycin, gentamicin, mupirocin, mafenide, phenol, retapamulin, silver sulfadiazine, sulfacetamide, and sulfanilamide. In some embodiments, the disease is a mycosis. In some embodiments, the mycosis is candidiasis. In some embodiments, the microbicide is selected from the group consisting of borax, butenafine, butoconazole, ciclopirox, clotrimazole, clotrimazole/betamethasone dipropionate, econazole, gentian violet, ketoconazole, naftifine, nystatin, nystatin/triamcinolone, oxiconazole, quaternary ammonium salts, silane quaternary ammonium salts, 3-(trihydroxysilyl) propyldimethyloctadecyl ammonium chloride, selenium sulfide, pyrithione zinc, miconazole, miconazole/zine oxide/petrolatum, tolnaftate, terbinafine, tioconazole, sertaconazole, sulconazole, and terbinafine. In some embodiments, the candidiasis is caused by a fungi species selected from the group consisting of Candida albicans, Candida glabrata, and Candida tropicalis. In some embodiments, the commensal organism is selected from the group consisting of Actinomyces, Bacteroides, Bifidobacterium, Clostridium, Corynebacterium, Enterococcus, Enterobacteriaceae, Eubacterium, Fusobacterium, Gardnerella, Haemophilus, Lactobacillus, Mobiluncus, Mycoplasma, Peptostreptococcus, Propionibacterium, Porphyromonas, Prevotella, Staphylococcus, Streptococcus, Treponema, and Ureaplasma. In some embodiments, the commensal organism is selected from the group consisting of Acinetobacter, Aerococcus, Bacillus, Clostridium, Corynebacterium, Micrococcus, Peptostreptococcus, Propionibacterium, Staphylococcus, and Streptococcus. In some embodiments, the commensal organism is selected from the group consisting of Acinetobacter, Actinomyces, Bacteroides, Bifidobacterium, Campylobacter, Clostridium, Corynebacterium, Eubacterium, Enterobacteriaceae, Enterococcus, Fusobacterium, Propionibacterium, Haemophilus, Helicobacter; Lactobacillus, Mobiluncus, Peptostreptococcus, Porphyromonas, Prevotella, Pseudomonas, Staphylococcus, and Streptococcus.

Some embodiments are directed to methods of treating vulvovaginal candidiasis, comprising topically administering an antimycotic to a tissue surface at a concentration sufficient to kill Candida albicans and at a concentration below the minimal inhibitory concentration for growth of at least one commensal organism. In some embodiments, the antimycotic is selected from the group consisting of borate salt, clotrimazole, oxiconazole, miconazole, ketoconazole, econazole, butoconazole, tioconazole, sertaconazole, and sulconazole. In some embodiments, the infection is a sequela of diabetes. In some embodiments, the topical administration is by means of a wipe.

Some embodiments are directed to methods of reducing the risk of infection in a patient; comprising wiping said patients urogenital region with a cleansing article after urination.

In some embodiments, the patient has glycosuria. In some embodiments, the patient is diabetic. In some embodiments, the patient is a female. In some embodiments, the patient is being treated with sulfonylureas, meglitinides, biguanides, thiazolidinediones, alpha-glucosidase inhibitors, sodium-glucose co-transporter-2 (SGLT2) inhibitors, and dipeptidyl peptidase-4 (DPP-4 inhibitors), or a combination thereof. In some embodiments, the DPP-4 inhibitor is selected from itagliptin, vildagliptin, saxagliptin, linagliptin, dutogliptin, gemigliptin, alogliptin, berberine and combinations thereof. In some embodiments, the SGLT2 inhibitor is selected from canagliflozin, dapagliflozin, ipragliflozin, empagliflozin, tofogliflozin, and combinations thereof. In some embodiments, the method is performed each time the patient urinates. In some embodiments, the method is performed for a period of time corresponding to the treatment with the diabetic treatments disclosed herein. In some embodiments, the method is carried out if the patient is diagnosed with glycosuria.

In some embodiments, the infection is a yeast infection. In some embodiments, the infection is diabetic recurrent vulvovaginal candidiasis. In some embodiments, the infection is vaginitis. In some embodiments the infection in balinitis.

In some embodiments, the cleansing article comprises a substrate; and a cleansing composition; wherein the cleansing composition is incorporated into the substrate. In some embodiments, the substrate is a fibrous substrate. In some embodiments, the fibrous substrate is a non-woven fabric. In some embodiments, the wherein the substrate comprises paper tissue, toilet paper, cellulose, polyvinyl alcohol, guar gum, starch, a polymer, or any combinations thereof. In some embodiments, the substrate is a wipe. In some embodiments, the cleansing composition comprises a wetting composition. In some embodiments, the wetting composition comprises a polyol, a lower level alcohol, a glycol, a ketone, or any combination thereof.

In some embodiments, the cleansing composition comprises a sequestering agent. In some embodiments, the sequestering agent is a glucose sequestering agent. In some embodiments, the glucose sequestering agent is selected from a zeolite, aluminum oxide microspheres, ceramic microspheres, hydrous alumina silicate microspheres, alumina dessicant beads, attapulgus clay beaded silica gel desiccants, natural clay absorbents, activated carbon or any combination thereof. In some embodiments, the cleansing agent is an aqueous cleansing composition.

In some embodiments, the cleansing composition comprises at least one cleansing surfactant. In some embodiments, the surfactant is present in an effective amount to provide a cleansing benefit. In some embodiments, the cleansing surfactant is an anionic surfactant, a nonionic surfactant, an amphoteric surfactant, a cationic surfactant, or any combination thereof.

In some embodiments, wherein the cleansing composition comprises a conditioning agent.

In some embodiments, the conditioning agent is a lipophilic conditioning agent. In some embodiments, the cleansing composition comprises a secondary active ingredient selected from a non-steroidal anti-inflammatory agent, a topical anesthetic, and a combination thereof.

In some embodiments, the cleansing composition comprises an antibiotic agent. In some embodiments, the concentration of the antibiotic agent in the cleansing composition is sufficient to kill at least one pathogenic microorganism and at a concentration below the minimal inhibitory concentration for growth of at least one commensal organism. In some embodiments, the antibiotic agent is selected from β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amantadine sulfate, octopirox, parachlorometa xylenol, nystatin, tolnaftate, zinc pyrithione and clotrimazole and combinations thereof.

In some embodiments, the cleansing composition comprises an microbicidal agent. In some embodiments, the concentration of the microbicidal agent in the cleansing composition is sufficient to kill at least one pathogenic microorganism and at a concentration below the minimal inhibitory concentration for growth of at least one commensal organism. In some embodiments, the microbicidal agent is selected from bacitracin, bacitracin/polymyxin B; bacitracin/neomycin/polymyxin B, neomycin/polymyxin B/hydrocortisone, bacitracin/neomycin/polymyxin B/hydrocortisone, benzalkonium chloride, benzalkonium chloride/pramoxine, benzalkonium chloride/lidocaine, benzethonium, benzethonium chloride/benzocaine, benzethonium chloride/lidocaine, borax, chlorhexidine, clindamycin, gentamicin, mupirocin, mafenide, phenol, retapamulin, silver sulfadiazine, sulfacetamide, sulfanilamide, borax, butenafine, butoconazole, ciclopirox, clotrimazole, clotrimazole/betamethasone dipropionate, econazole, gentian violet, ketoconazole, naftifine, nystatin, nystatin/triamcinolone, oxiconazole, quaternary ammonium salts, silane quaternary ammonium salts, 3-(trihydroxysilyl) propyldimethyloctadecyl ammonium chloride, selenium sulfide, pyrithione zinc, miconazole, miconazole/zine oxide/petrolatum, tolnaftate, terbinafine, tioconazole, sertaconazole, sulconazole, terbinafine and combinations thereof.

In some embodiments, the cleansing composition comprises an antimycotic agent. In some embodiments, the concentration of the antimycotic agent is the cleansing composition is sufficient to kill at least one pathogenic microorganism and at a concentration below the minimal inhibitory concentration for growth of at least one commensal organism. In some embodiments, the antimycotic agent is selected from borate salt, clotrimazole, oxiconazole, miconazole, ketoconazole, econazole, butoconazole, tioconazole, sertaconazole, sulconazole, and combinations thereof.

This invention and embodiments illustrating the method and materials used may be further understood by reference to the following non-limiting examples.

### Example 1

A wipe is manufactured with a composition comprising pramoxine hydrochloride 1% having inactive ingredients include polysorbate 20, glycerin, phenoxyethanol, dosodium cocoamphodiacetate, TEA-cocoyl Glutamate, methylparaen, ethylparaben, disodium EDTA, PEG-7 Glyceryl Cocoate, aloe barbadensis leaf juice, tocopherol (vitamine E) acetate, Zea mays (corn) oil, retinyl (vitamin A) palmitate, cholecalciferol (Vitamin D3).

### Example 2

A wipe is manufactured comprising PEG-40, hydrogenated castor oil, benzyl alcohol, iodopropynyl butylcarbamate, bis-PEG/GGP-16/16 PEG/PPG-1616 dimethicone, caprylic/capric triglyceride, xanthan gum, disodium EDTA, sodium hydroxymethylglycinate, and citric acid.

### Example 3

A wipe is manufactured comprising benzocaine 5% and resorcinol 2% as topical analgesics.

### Example 4

A wipe is manufactured comprising, borax (sodium borate), cuprum sulph (copper sulfate), zincum sulph (zinc sulfate), carbolicum (carbolic acid), arg met 8× (metallic silver), calc carb 12× (oyster shells), kali bic (potassium bichromate), and sepia.

Although the present invention has been described in considerable detail with reference to certain preferred embodiments thereof, other versions are possible. Therefore, the spirit and scope of the appended claims should not be limited to the description and the preferred embodiments disclosed herein.

1.-24. (canceled)

25. A wipe comprising:
a nonwoven fabric;
at least one gripping feature defined within the fabric, the at least one gripping feature configured to receive at least a portion of a hand of a user of the wipe; and
one or more creases defining at least one fold, wherein the wipe is configured to be folded on the one or more creases prior to being inserted into a package.

26. The wipe of claim 25, wherein the nonwoven fabric is flushable nonwoven fabric.

27. The wipe of claim 26, wherein the at least one gripping feature comprising a pocket.

28. The wipe of claim 27, wherein the pocket comprises the nonwoven fabric.

29. The wipe of claim 28, wherein the pocket is flushable.

30. The wipe of claim 25, wherein the nonwoven fabric is dry prior to being inserted into the package.

31. The wipe of claim 25, wherein the nonwoven fabric is configured to absorb a cleansing fluid.

32. The wipe of claim 31, wherein the package is configured to hold at least one burst pod, the burst pod configured to release the cleansing fluid prior to the package being opened.

33. The wipe of claim 32, wherein the cleansing fluid is a cleanser suitable for use on human skin.

34. The wipe of claim 31, wherein the cleansing fluid comprises at least one oxido-reductase enzyme.

35. The wipe of claim 34, wherein the cleansing fluid comprises glucose oxidase.

36. A package comprising:
a burst pod at least partially attached to the package and configured to store a cleansing fluid; and
a single-use wipe positioned adjacent to at least a portion of the burst pod,
wherein the burst pod is configured to rupture upon application of a specific pressure, thereby releasing the cleansing fluid onto the wipe;
wherein the cleansing fluid comprises at least one oxido-reductase enzyme;
wherein the cleansing fluid and single-use wipe are free from glucose; and
wherein the burst pod comprises multiple exit ports configured to direct the flow of the cleansing fluid to the single-use wipe.

37. The package of claim 36, wherein the single-use wipe comprises:
a nonwoven fabric;
at least one gripping feature defined within the fabric, the at least one gripping feature configured to receive at least a portion of a hand of a user of the wipe; and
one or more creases defining at least one fold, wherein the wipe is configured to be folded on the one or more creases prior to being inserted into the package.

38. The package of claim 37, wherein the nonwoven fabric is flushable nonwoven fabric.

39. The package of claim 38, wherein the at least one gripping feature comprises a pocket.

40. The package of claim 39, wherein the pocket comprises the nonwoven fabric.

41. The package of claim 40, wherein the pocket is flushable.

42. The package of claim 37, wherein the nonwoven fabric is dry prior to being inserted into the package.

43. The package of claim 37, wherein the nonwoven fabric is configured to absorb the cleansing fluid.

44. The package of claim 36, wherein the cleansing fluid is a cleanser suitable for use on human skin.

45. The package of claim 36, wherein the cleansing fluid comprises glucose oxidase.

46. The package of claim 36, further comprising at least one opening mechanism.

47. The package of claim 46, wherein the at least one opening mechanism comprises at least one of a liftable flap and a tearaway strip.
